(19) United States
(12) Patent Application Publication
(10) Pub. No.: US 2026/0217860 A1
(43) Pub. Date: Jul. 30, 2026

(54) ANTI-EGFR/MUC1 ANTIBODIES AND USES THEREOF

(71) Applicant: Biocytogen Pharmaceuticals (Beijing) Co., Ltd., Beijing (CN)

(72) Inventors: Yifu Zhang, Beijing (CN); Yanfei Han, Beijing (CN); Zhuolin Li, Beijing (CN); Chengzhang Shang, Beijing (CN); Yi Yang, Beijing (CN); Yuelei Shen, Beijing (CN)

(21) Appl. No.: 19/146,348

(22) PCT Filed: Jan. 8, 2024
(86) PCT No.: PCT/CN2024/071122

§ 371 Date: Jul. 8, 2025

(30) Foreign Application Priority Data
Jan. 9, 2023 (WO) PCT/CN2023/071272

(51) Int. Cl. C07K 16/30; A61K 39/00; A61K 45/06; A61K 47/68; A61P 35/00; C07K 16/28

(52) U.S. Cl. CPC C07K 16/3092; A61K 45/06; A61K 47/68031; A61K 47/6879; A61K 47/6889; A61P 35/00; C07K 16/2818; C07K 16/2863; A61K 2039/507; C07K 2317/14; C07K 2317/24; C07K 2317/31; C07K 2317/33; C07K 2317/565; C07K 2317/622; C07K 2317/77

(57) ABSTRACT

It relates to anti-EGFR/MUC1 antibodies, and antibody drug conjugates derived therefrom, and the use thereof.

### CLAIM OF PRIORITY

This application claims priority to PCT/CN2023/071272, filed on Jan. 9, 2023. The entire contents of the foregoing application are incorporated herein by reference.

### TECHNICAL FIELD

This disclosure relates to multispecific anti-EGFR (epidermal growth factor receptor)/MUCt (mucin 1) antibodies (e.g., bispecific antibodies or antigen-binding fragments thereof), and antibody drug conjugates derived therefrom.

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications, there is a need to continue to develop various therapeutics based on bispecific antibodies.

### SUMMARY

This disclosure relates to anti-EGFR/MUC1 antibodies or antigen-binding fragments thereof, wherein the antibodies or antigen-binding fragments thereof specifically bind to EGFR and MUC1. In some embodiments, the antibodies or antigen-binding fragments thereof have identical light chain variable regions. In some embodiments, the antibodies or antigen-binding fragments thereof have a common light chain. The disclosure also relates to antibody drug conjugates derived from these anti-EGFR/MUC1 antibodies.

In one aspect, the disclosure is related to an anti-EGFR/MUC1 antibody or antigen-binding fragment thereof, comprising: a first antigen-binding domain that specifically binds to EGFR; and a second antigen-binding domain that specifically binds to MUC1.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and

the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,

wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively; and
(10) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and

the second light chain variable region (VL2) comprises CDRs 1, 2, and 3, wherein the VL2 CDR 1 region comprises an amino acid sequence that is at least 80% identical to a selected VL2 CDR 1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,

wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively; and
(4) the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

In some embodiments, one of the following is true: (1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(18) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(19) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively; or
(20) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

In some embodiments, the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

In some embodiments, the VH1 comprises an amino acid sequence that is at least 90% identical to a selected VH sequence, and the VL 1 comprises an amino acid sequence that is at least 90% identical to a selected VL sequence, wherein the selected VH sequence and the selected VL sequence are one of the following:
(1) the selected VH sequence is SEQ ID NO: 47, and the selected VL sequence is SEQ ID NO: 46;
(2) the selected VH sequence is SEQ ID NO: 48, and the selected VL sequence is SEQ ID NO: 46;
(3) the selected VH sequence is SEQ ID NO: 49, and the selected VL sequence is SEQ ID NO: 46;
(4) the selected VH sequence is SEQ ID NO: 50, and the selected VL sequence is SEQ ID NO: 46; and
(5) the selected VH sequence is SEQ ID NO: 51, and the selected VL sequence is SEQ ID NO: 46.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 47, and the selected VL sequence is SEQ ID NO: 46;
(2) the selected VH sequence is SEQ ID NO: 48, and the selected VL sequence is SEQ ID NO: 46;
(3) the selected VH sequence is SEQ ID NO: 49, and the selected VL sequence is SEQ ID NO: 46;
(4) the selected VH sequence is SEQ ID NO: 50, and the selected VL sequence is SEQ ID NO: 46; and
(5) the selected VH sequence is SEQ ID NO: 51, and the selected VL sequence is SEQ ID NO: 46.

In some embodiments, the VH2 comprises an amino acid sequence that is at least 90% identical to a selected VH sequence, and the VL2 comprises an amino acid sequence that is at least 90% identical to a selected VL sequence, wherein the selected VH sequence and the selected VL sequence are one of the following:
(1) the selected VH sequence is SEQ ID NO: 52, and the selected VL sequence is SEQ ID NO: 46; and
(2) the selected VH sequence is SEQ ID NO: 53, and the selected VL sequence is SEQ ID NO: 46.

In some embodiments, the VH2 comprises VH2 CDR1, VH2 CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 52, and the selected VL sequence is SEQ ID NO: 46; and
(2) the selected VH sequence is SEQ ID NO: 53, and the selected VL sequence is SEQ ID NO: 46.

In some embodiments, the VH1 comprises the sequence of SEQ ID NO: 47 and the VL1 comprises the sequence of SEQ ID NO: 46.

In some embodiments, the VH1 comprises the sequence of SEQ ID NO: 48 and the VL1 comprises the sequence of SEQ ID NO: 46.

In some embodiments, the VH1 comprises the sequence of SEQ ID NO: 49 and the VL1 comprises the sequence of SEQ ID NO: 46.

In some embodiments, the VH1 comprises the sequence of SEQ ID NO: 50 and the VL1 comprises the sequence of SEQ ID NO: 46.

In some embodiments, the VH1 comprises the sequence of SEQ ID NO: 51 and the VL1 comprises the sequence of SEQ ID NO: 46.

In some embodiments, the VH2 comprises the sequence of SEQ ID NO: 52 and the VL2 comprises the sequence of SEQ ID NO: 46.

In some embodiments, the VH2 comprises the sequence of SEQ ID NO: 53 and the VL2 comprises the sequence of SEQ ID NO: 46.

In some embodiments, the first antigen-binding domain specifically binds to human or monkey EGFR; and/or the second antigen-binding domain specifically binds to human or monkey MUC1.

In some embodiments, the first antigen-binding domain is human or humanized; and/or the second antigen-binding domain is human or humanized.

In some embodiments, the antibody is a bispecific antibody.

In some embodiments, the first antigen-binding domain is a single-chain variable fragment (scFv); and/or the second antigen-binding domain is a scFv.

In some embodiments, the first light chain variable region and the second light chain variable region are identical.

In some embodiments, the disclosure is related to an anti-EGFR/MUC1 antibody or antigen-binding fragment thereof that cross-competes with the anti-EGFR/MUC1 antibody or antigen-binding fragment thereof described herein.

In one aspect, the disclosure is related to a nucleic acid comprising a polynucleotide encoding the anti-EGFR/MUC1 antibody or antigen-binding fragment thereof described herein.

In one aspect, the disclosure is related to a vector comprising the nucleic acid described herein.

In one aspect, the disclosure is related to a cell comprising the vector described herein.

In some embodiments, the cell is a CHO cell.

In one aspect, the disclosure is related to a cell comprising the nucleic acid described herein.

In one aspect, the disclosure is related to a method of producing an anti-EGFR/MUC1 antibody or an antigen-binding fragment thereof, the method comprising
(a) culturing the cell described herein under conditions sufficient for the cell to produce the anti-EGFR/MUC1 antibody or the antigen-binding fragment thereof; and
(b) collecting the anti-EGFR/MUC1 antibody or the antigen-binding fragment thereof produced by the cell.

In one aspect, the disclosure is related to an anti-EGFR/MUC1 antibody-drug conjugate (ADC) comprising a therapeutic agent covalently bound to the anti-EGFR/MUC1 antibody or antigen-binding fragment thereof described herein.

In some embodiments, the therapeutic agent is a cytotoxic or cytostatic agent.

In some embodiments, the therapeutic agent is MMAE or MMAF.

In some embodiments, the therapeutic agent is selected from:

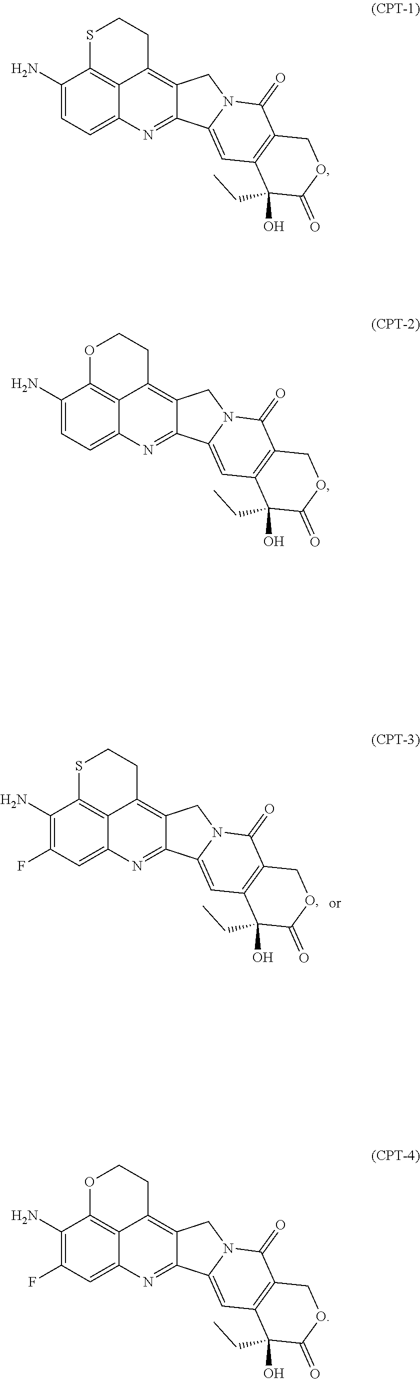 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

In some embodiments, the therapeutic agent is linked to the antibody or antigen-binding fragment thereof via a linker. In some embodiments, the linker has a structure of:

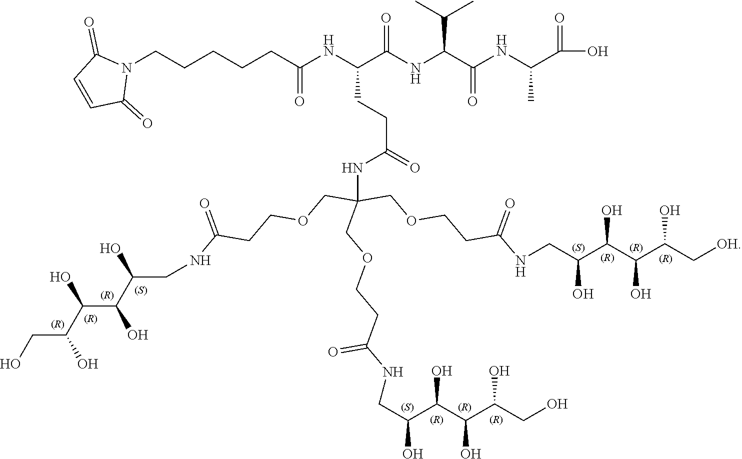

In some embodiments, the antibody-drug conjugate has a structure of:

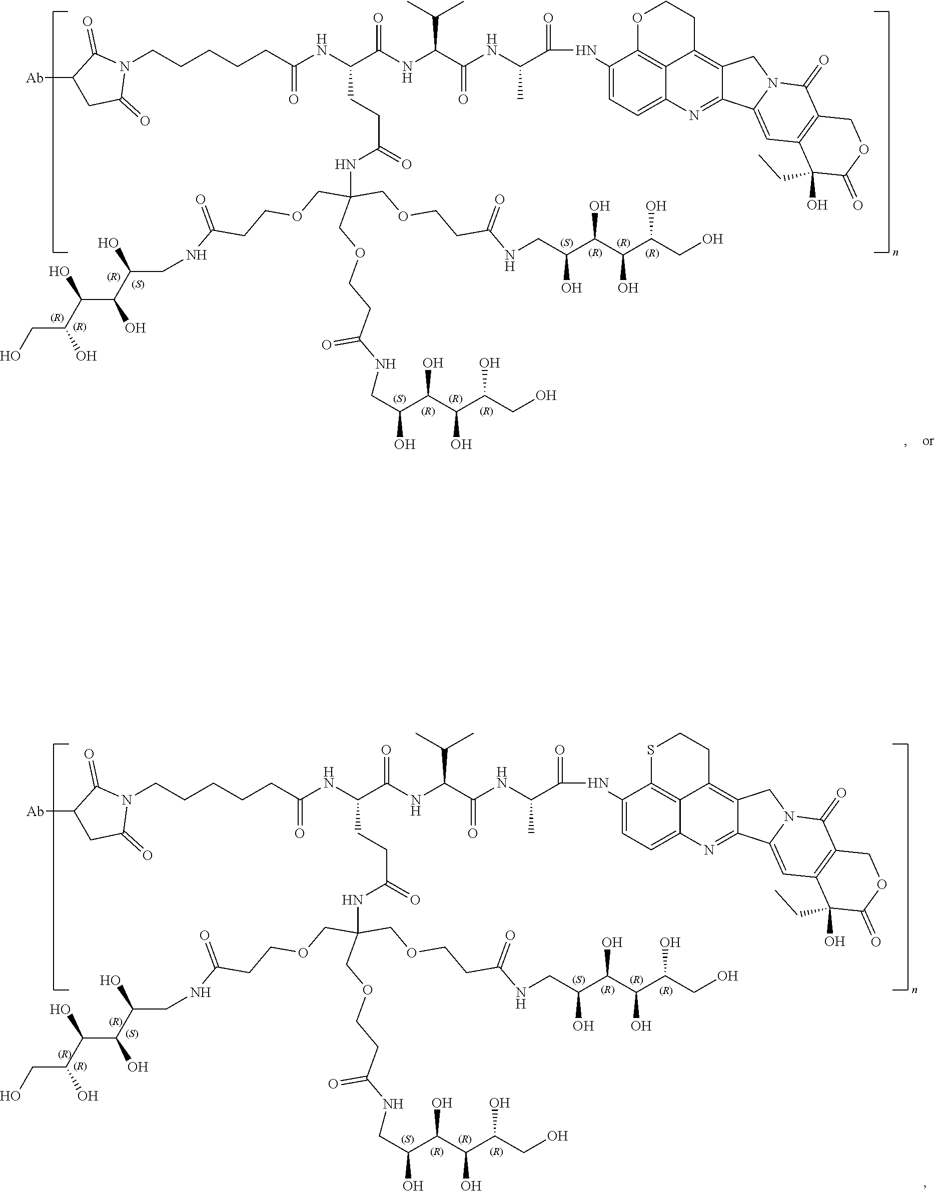

in some embodiments, n=1-8; in some embodiments, “Ab” represents the antibody or antigen-binding fragment thereof.

In some embodiments, the drug-to-antibody ratio (DAR) is about 4 or 8.

In one aspect, the disclosure is related to a method of treating a subject having cancer, the method comprising administering a therapeutically effective amount of a composition comprising the anti-EGFR/MUC1 antibody or antigen-binding fragment thereof described herein, or the anti-EGFR/MUC1 antibody-drug conjugate described herein, to the subject.

In some embodiments, the subject has a cancer expressing EGFR and/or MUC1 (e.g., both EGFR and MUC1).

In some embodiments, the cancer is solid tumor, lung cancer, head and neck cancer, colon cancer, pancreatic cancer, breast cancer, ovarian cancer, bladder cancer, kidney cancer, gliomas, oesophageal cancer, gastric cancer, stomach cancer, prostate cancer, cervical cancer, multiple myeloma, or Non-Hodgkin Lymphoma.

In some embodiments, the subject is a human.

In some embodiments, the method further comprises administering an anti-PD1 antibody to the subject.

In some embodiments, the method further comprises administering a chemotherapy to the subject.

In one aspect, the disclosure is related to a method of decreasing the rate of tumor growth, the method comprising contacting a tumor cell with an effective amount of a composition comprising the anti-EGFR/MUC1 antibody or antigen-binding fragment thereof described herein, or the anti-EGFR/MUC1 antibody-drug conjugate described herein.

In one aspect, the disclosure is related to a method of killing a tumor cell, the method comprising contacting a tumor cell with an effective amount of a composition comprising the anti-EGFR/MUC1 antibody or antigen-binding fragment thereof described herein, or the anti-EGFR/MUC1 antibody-drug conjugate described herein.

In one aspect, the disclosure is related to a pharmaceutical composition comprising a pharmaceutically acceptable carrier and (a) the anti-EGFR/MUC1 antibody or antigen-binding fragment thereof described herein, and/or (b) the anti-EGFR/MUC1 antibody-drug conjugate described herein.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or control antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a control antigen). In other examples, an antigen-binding domain can bind to two different antigens (e.g., an effector antigen and a control antigen).

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, bispecific antibodies, and multispecific antibodies.

One example of an antibody is a protein complex that includes two heavy chains and two light chains. Additional examples of an antibody are described herein.

As used herein, the term “multispecific antibody” is an antibody that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multispecific antibody binds two different epitopes (i.e., a “bispecific antibody”). In some aspects, a multispecific antibody binds three different epitopes (i.e., a “trispecific antibody”). In some aspects, a multispecific antibody binds four different epitopes (i.e., a “quadspecific antibody”). In some aspects, a multispecific antibody binds five different epitopes (i.e., a “quintspecific antibody”). Each binding specificity may be present in any suitable valency. Non-limiting examples of multispecific antibodies are described herein.

As used herein, the term “bispecific antibody” refers to an antibody that binds to two different epitopes. The epitopes can be on the same antigen or on different antigens.

As used herein, the term “common light chain” refers to a light chain that can interact with two or more different heavy chains, forming different antigen-binding sites, wherein these different antigen-binding sites can specifically bind to different antigens or epitopes. Similarly, the term “common light chain variable region” refers to a light chain variable region that can interact with two or more different heavy chain variable regions, forming different antigen-binding sites, wherein these different antigen-binding sites can specifically bind to different antigens or epitopes. In some embodiments, the antibody or antigen-binding fragment thereof can have a common light chain. In some embodiments, the anti-EGFR/MUC1 antibody or antigen-binding fragment thereof can have a common light chain variable region.

As used herein, the term “anti-EGFR/MUC1 antibody or antigen-binding fragment thereof” refers to an antibody or antigen-binding fragment that binds to both MUC1 and EGFR.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows an exemplary structure of the anti-EGFR/MUC1 antibody described herein.

FIG. 2 shows the average tumor volumes in different groups of B-NDG mice that were injected with HCC70 cells, and were treated with phosphate buffer saline (PBS) or ADCs.

FIG. 3 shows the average tumor volumes in different groups of B-NDG mice that were injected with Panc 02.03 cells, and were treated with PBS or ADCs.

FIG. 4 shows the average tumor volumes in different groups of B-NDG mice that were injected with NCI-H1975 cells, and were treated with PBS or ADCs.

FIG. 5 shows the average tumor volumes in different groups of B-NDG mice that were injected with NCI-H1650 cells, and were treated with PBS or ADCs.

FIG. 6 lists heavy chain variable region CDR sequences of anti-EGFR antigen binding domain (E-6C4, E-9A6, E-9D2, E-9F3 and E-9H2) and an anti-MUC1 antigen binding domain (10M1 and M-1 IE8) in anti-EGFR/MUC1 antibodies as defined by Kabat definition.

FIG. 7 lists heavy chain variable region CDR sequences of anti-EGFR antigen binding domain (E-6C4, E-9A6, E-9D2, E-9F3 and E-9H2) and an anti-MUC1 antigen binding domain (10M1 and M-11E8) in anti-EGFR/MUC1 antibodies as defined by Chothia definition.

FIG. 8 lists light chain variable region CDR sequences of anti-EGFR/MUC1 antibodies as defined by Kabat definition and Chothia definition.

FIG. 9 lists the heavy chain variable region and light chain variable region sequences of anti-EGFR/MUC1 antibodies.

FIG. 10 lists certain amino acid sequences discussed in the disclosure.

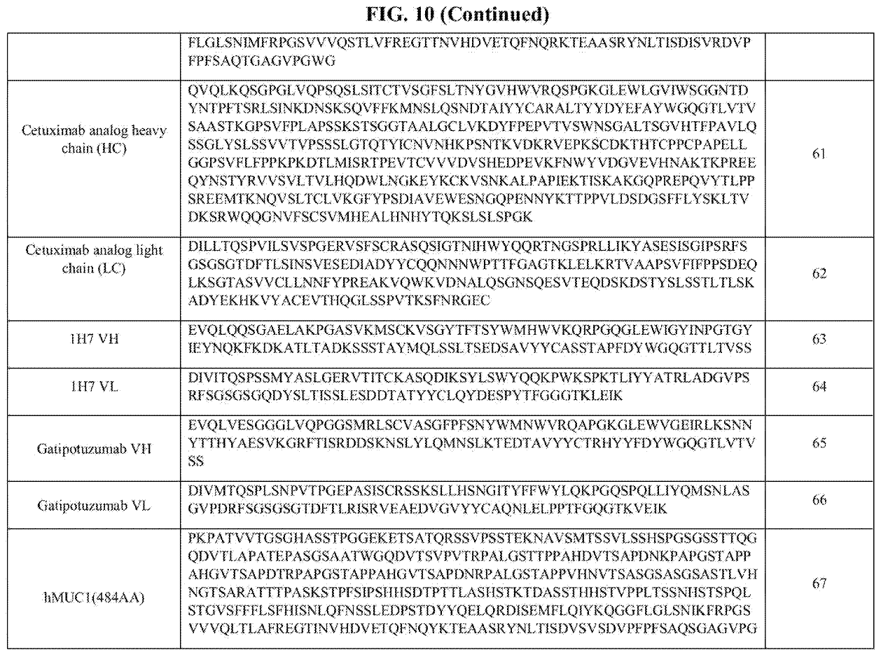

FIG. 11 shows the average tumor volume in different groups of B-NDG mice that were engrafted with patient-derived ampullary tumor fragments, and were treated with PBS or ADCs.

FIGS. 12A-12B show the endocytosis rates of the anti-EGFR antibody, anti-MUC1 antibody and anti-EGFR/MUC1 bispecific antibody in NUGC-4 cells (FIG. 12A) or HCC70 cells (FIG. 12B).

FIGS. 13A-13B show the endocytosis rates of the anti-EGFR/MUC1 bispecific antibody and ADC in NUGC-4 cells (FIG. 13A) or Pane 02.03 cells (FIG. 13B).

FIG. 14 shows the average tumor volume in different groups of B-NDG mice that were engrafted with patient-derived pancreatic tumor fragments, and were treated with PBS or ADCs.

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms.

Each arm can have one heavy chain variable region and one light chain variable region, forming an antigen-binding domain (or an antigen-binding region). In some embodiments, the bispecific antibody has a common light chain.

The present disclosure relates to anti-EGFR/MUC1 antibodies (e.g., bispecific antibodies or antigen-binding fragments thereof) that specifically bind to EGFR and MUC1, and antibody drug conjugates derived from these anti-EGFR/MUC1 antibodies.

Anti-EGFR/MUC1 Antibody Epidermal growth factor receptor (EGFR, ErbBI or HER1) is a Type I transmembrane glycoprotein of 170 kDa that is encoded by the c-erbBl proto-oncogene. The epidermal growth factor receptor is a member of the ErbB family of receptors, a subfamily of four closely related receptor tyrosine kinases: EGFR (ErbB-1), HER2/neu (ErbB-2), Her3 (ErbB-3) and Her4 (ErbB-4). In many cancer types, mutations affecting EGFR expression or activity could result in cancer. EGFR signaling is initiated by ligand binding followed by induction of conformational change, homodimerization or heterodimerization of the receptor with other ErbB family members, and trans-autophosphorylation of the receptor, which initiates signal transduction cascades that ultimately affect a wide variety of cellular functions, including cell proliferation and survival, increases in expression or kinase activity of EGFR have been linked with a range of human cancers, making EGFR an attractive target for therapeutic intervention. Increases in both the EGFR gene copy number and protein expression have been associated with favorable responses to the EGFR tyrosine kinase inhibitor, IRESSA™ (gefitinib), in non-small cell lung cancer.

Binding of a ligand such as EGF (epidermal growth factor) to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division.

Inhibition of EGFR signaling may result in inhibition in one or more EGFR. In some embodiments, the EGFR ligands include EGF, TGFα, heparin binding EGF (HB-EGF), amphiregulin (AR), and epiregulm (EPI).

A detailed review of EGFR can be found in Sabbah, Dima A., Rima Hajjo, and Kamal Sweidan. “Review on epidermal growth factor receptor (EGFR) structure, signaling pathways, interactions, and recent updates of EGFR inhibitors.” Current topics in medicinal chemistry (2020); which is incorporated herein by reference in its entirety.

Mucin 1 (MUC1; also known as episialin, PEM, H23Ag, EMA, CA15-3, and MCA) is a single pass type I transmem-brane protein with a heavily glycosylated extracellular domain that extends up to 200-500 nm from the cell surface. MUC1 is normally expressed in the glandular or luminal epithelial cells of the mammary gland, esophagus, stomach, duodenum, pancreas, uterus, prostate, and lungs, and to a lesser extent, in hematopoi-etic cells. It is absent in the skin epithelium and in mesenchymal cells. In healthy tissues, MUC1 provides protection to the underlying epithelia. The extended negatively charged sugar branches of MUC1 create a physical barrier and impart an anti-adhesive property to MUC1, limiting accessibility and preventing pathogenic colonization. The sugar chains oligomerize to form a mucinous gel that lubricates and protects the underlying epithelia from desiccation, changes in pH, pollutants, and microbes. Aberrantly glycosylated MUC1 is overexpressed in most human epithelial cancers and has gained remarkable attention as an oncogenic molecule.

MUC1 is overexpressed in cancer cells and the loss of cell polarity causes TA-MUC1 to be redistributed over the cell surface and within the cytoplasm. Lack of cell polarity also causes the redistribution of cell surface growth factors that are normally restricted to the basolateral surface of epithelial cells. Growth factors juxtaposed to MUC1 and intracellular kinases such as ZAP-70, PKC-g, GSK-3b, and c-Src phosphorylate serine, tyrosine, and threonine residues on MUC1 CT. It is also thought that hypoglycosylation unmasks the peptide core of TA-MUC1 allowing MUC1-N cleavage and release by extracellular proteases. MUC1-N release induces conformational changes in MUC1-C that alter its ligand status and subsequently activates downstream cell signaling pathways such as the mitogen-activated protein kinase (MAPK), phosphatidylinositol 3-kinase (P13K/Akt), and wingless type (Wnt) pathways. As a result, MUC1—positive pancreas, breast, lung, and colon cancer cells commonly display hyperactivation of these critical signal-ing pathways. MUC1-C also associates with various transcription factors (STAT3, NF-kB, p53, and b-catenin) and binds the target gene promoter region to drive their expression. Several studies have indicated that MUC1 plays a critical role in the transcriptional regulation of genes associated with tumor invasion, metastasis, angiogenesis, proliferation, apoptosis, drug resis-tance, inflammation, and immune regulation.

A detailed review of MUC1 and its functions can be found in Nath, Sritama, and Pinku Mukherjee. “MUC1: a multifaceted oncoprotein with a key role in cancer progression.” Trends in molecular medicine 20.6 (2014): 332-342, which is incorporated by reference in its entirety.

In some embodiments, the bispecific anti-EGFR/MUC1 antibody described herein can be designed to have an IgG1 subtype structure with knobs-into-holes (KIH) mutations, which can promote heterodimerization and avoid mispairing between the two heavy chains. In some embodiments, the bispecific anti-EGFR/MUC1 antibody has a higher endocytosis rate than the corresponding monoclonal antibodies or the control bispecific antibodies.

In some embodiments, the bispecific anti-EGFR/MUC1 antibody described herein can be conjugated with a therapeutic agent, forming an antibody drug conjugate (ADC). In some embodiments, the drug-to-antibody ratio (DAR) of the ADCs described herein is about 3.8, about 3.9, about 4.0, about 4.1, about 4.2, about 4.3, about 4.4, about 4.5, about 4.6, or about 4.7. In some embodiments, the DAR of the ADCs described herein is about 3.5 to about 4.5, about 3.6 about 4.5, about 3.7 to about 4.5, about 3.8 to about 4.5, about 3.9 to about 4.5, about 4.0 to about 4.5, about 4.1 to about 4.5, about 4.2 to about 4.5, about 4.3 to about 4.5, about 4.4 to about 4.5, about 3.5 to about 4.4, about 3.6 to about 4.4, about 3.7 to about 4.4, about 3.8 to about 4.4, about 3.9 to about 4.4, about 4.0 to about 4.4, about 4.1 to about 4.4, about 4.2 to about 4.4, about 4.3 to about 4.4, about 3.5 to about 4.3, about 3.6 to about 4.3, about 3.7 to about 4.3, about 3.8 to about 4.3, about 3.9 to about 4.3, about 4.0 to about 4.3, about 4.1 to about 4.3, about 4.2 to about 4.3, about 3.5 to about 4.2, about 3.6 to about 4.2, about 3.7 to about 4.2, about 3.8 to about 4.2, about 3.9 to about 4.2, about 4.0 to about 4.2, about 4.1 to about 4.2, about 3.5 to about 4.1, about 3.6 to about 4.1, about 3.7 to about 4.1, about 3.8 to about 4.1, about 3.9 to about 4.1, about 4.0 to about 4.1, about 3.5 to about 4.0, about 3.6 to about 4.0, about 3.7 to about 4.0, about 3.8 to about 4.0, about 3.9 to about 4.0, about 3.5 to about 3.9, about 3.6 to about 3.9, about 3.7 to about 3.9, about 3.8 to about 3.9, about 3.5 to about 3.8, about 3.6 to about 3.8, about 3.7 to about 3.8, about 3.5 to about 3.7, about 3.6 to about 3.7, or about 3.5 to about 3.6. In some embodiments, the DAR of the ADCs described herein is about 7.5 to about 8.5, about 7.6 to about 8.5, about 7.7 to about 8.5, about 7.8 to about 8.5, about 7.9 to about 8.5, about 8.0 to about 8.5, about 8.1 to about 8.5, about 8.2 to about 8.5, about 8.3 to about 8.5, about 8.4 to about 8.5, about 7.5 to about 8.4, about 7.6 to about 8.4, about 7.7 to about 8.4, about 7.8 to about 8.4, about 7.9 to about 8.4, about 8.0 to about 8.4, about 8.1 to about 8.4, about 8.2 to about 8.4, about 8.3 to about 8.4, about 7.5 to about 8.3, about 7.6 to about 8.3, about 7.7 to about 8.3, about 7.8 to about 8.3, about 7.9 to about 8.3, about 8.0 to about 8.3, about 8.1 to about 8.3, about 8.2 to about 8.3, about 7.5 to about 8.2, about 7.6 to about 8.2, about 7.7 to about 8.2, about 7.8 to about 8.2, about 7.9 to about 8.2, about 8.0 to about 8.2, about 8.1 to about 8.2, about 7.5 to about 8.1, about 7.6 to about 8.1, about 7.7 to about 8.1, about 7.8 to about 8.1, about 7.9 to about 8.1, about 8.0 to about 8.1, about 7.5 to about 8.0, about 7.6 to about 8.0, about 7.7 to about 8.0, about 7.8 to about 8.0, about 7.9 to about 8.0, about 7.5 to about 7.9, about 7.6 to about 7.9, about 7.7 to about 7.9, about 7.8 to about 7.9, about 7.5 to about 7.8, about 7.6 to about 7.8, about 7.7 to about 7.8, about 7.5 to about 7.7, about 7.6 to about 7.7, or about 7.5 to about 7.6.

In some embodiments, the anti-EGFR/MUC1 ADC described herein can effectively inhibit in vitro cancer cell growth at a concentration of less than 10 μg/mL, less than 3.33 μg/mL, less than 1.11 μg/mL, less than 0.37 μg/mL, less than 0.12 μg/mL, less than 0.04 μg/mL, or less than 0.01 μg/mL. In some embodiments, the anti-EGFR/MUC1 ADC described herein can inhibit in vivo cancer cell growth (e.g., lung cancer, gastric cancer, breast cancer, or pancreatic cancer) in a xenograft mouse model at a dose level of less than 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

In some embodiments, the anti-EGFR/MUC1 antibody described herein has a common light chain. In some embodiments, the anti-EGFR/MUC1 antibody includes an anti-EGFR antigen-binding domain (e.g., E-6C4, E-9A6, E-9D2, E-9F3 or E-9H2) or an anti-MUC1 antigen-binding domain (e.g., 10M1 or M-1 1E8). In some embodiments, the anti-EGFR/MUC1 antibodies have a heavy chain variable region targeting EGFR (e.g., any one of the VH targeting EGFR described herein), a heavy chain variable region targeting MUC1 (e.g., any one of the VH targeting MUC1 described herein), and two identical common light chain variable regions.

The CDR sequences for E-6C4 antigen-binding domain include CDRs of the heavy chain variable domain, SEQ ID NOs: 4-6, and CDRs of the light chain variable domain, SEQ ID NOs: 1-3 as defined by Kabat definition. The CDRs can also be defined by Chothia definition. Under the Chothia definition, the CDR sequences of the heavy chain variable domain are set forth in SEQ ID NOs: 25-27, and CDR sequences of the light chain variable domain are set forth in SEQ ID NOs: 1-3. The human light chain variable region and human heavy chain variable region for E-6C4 are shown in SEQ ID NO: 46 and SEQ ID NO: 47, respectively.

The CDR sequences for E-9A6 antigen-binding domain include CDRs of the heavy chain variable domain, SEQ ID NOs: 7-9, and CDRs of the light chain variable domain, SEQ ID NOs: 1-3, as defined by Kabat definition. Under Chothia definition, the CDR sequences of the heavy chain variable domain are set forth in SEQ ID NOs: 28-30, and CDRs of the light chain variable domain are set forth in SEQ ID NOs: 1-3. The human light chain variable region and human heavy chain variable region for E-9A6 are shown in SEQ ID NO: 46 and SEQ ID NO: 48, respectively.

The CDR sequences for E-9D2 antigen-binding domain include CDRs of the heavy chain variable domain, SEQ ID NOs: 10-12, and CDRs of the light chain variable domain, SEQ ID NOs: 1-3, as defined by Kabat definition. Under Chothia definition, the CDR sequences of the heavy chain variable domain are set forth in SEQ ID NOs: 31-33, and CDRs of the light chain variable domain are set forth in SEQ ID NOs: 1-3. The human light chain variable region and human heavy chain variable region for E-9D2 are shown in SEQ ID NO: 46 and SEQ ID NO: 49, respectively.

The CDR sequences for E-9F3 antigen-binding domain include CDRs of the heavy chain variable domain, SEQ ID NOs: 13-15, and CDRs of the light chain variable domain, SEQ ID NOs: 1-3, as defined by Kabat definition. Under Chothia definition, the CDR sequences of the heavy chain variable domain are set forth in SEQ ID NOs: 34-36, and CDRs of the light chain variable domain are set forth in SEQ ID NOs: 1-3. The human light chain variable region and human heavy chain variable region for E-9F3 are shown in SEQ ID NO: 46 and SEQ ID NO: 50, respectively.

The CDR sequences for E-9H2 antigen-binding domain include CDRs of the heavy chain variable domain, SEQ ID NOs: 16-18, and CDRs of the light chain variable domain, SEQ ID NOs: 1-3, as defined by Kabat definition. Under Chothia definition, the CDR sequences of the heavy chain variable domain are set forth in SEQ ID NOs: 37-39, and CDRs of the light chain variable domain are set forth in SEQ ID NOs: 1-3. The human light chain variable region and human heavy chain variable region for E-9H2 are shown in SEQ ID NO: 46 and SEQ ID NO: 51, respectively.

The CDR sequences for 10M1 antigen-binding domain include CDRs of the heavy chain variable domain, SEQ ID NOs: 19-21, and CDRs of the light chain variable domain, SEQ ID NOs: 1-3, as defined by Kabat definition. Under Chothia definition, the CDR sequences of the heavy chain variable domain are set forth in SEQ ID NOs: 40-42, and CDRs of the light chain variable domain are set forth in SEQ ID NOs: 1-3. The human light chain variable region and human heavy chain variable region for 10M1 are shown in SEQ ID NO: 46 and SEQ ID NO: 52, respectively.

The CDR sequences for M-11E8 antigen-binding domain include CDRs of the heavy chain variable domain, SEQ ID NOs: 22-24, and CDRs of the light chain variable domain, SEQ ID NOs: 1-3, as defined by Kabat definition. Under Chothia definition, the CDR sequences of the heavy chain variable domain are set forth in SEQ ID NOs: 43-45, and CDRs of the light chain variable domain are set forth in SEQ ID NOs: 1-3. The human light chain variable region and human heavy chain variable region for M-11E8 are shown in SEQ ID NO: 46 and SEQ ID NO: 53, respectively.

In some embodiments, the anti-EGFR/MUC1 antibodies described herein can contain one, two, or three heavy chain variable region CDRs selected from the group of SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOs: 37-39, SEQ ID NO: 40-42, and SEQ ID NOs: 43-45; and/or one, two, or three light chain variable region CDRs set for in SEQ ID NOs: 1-3.

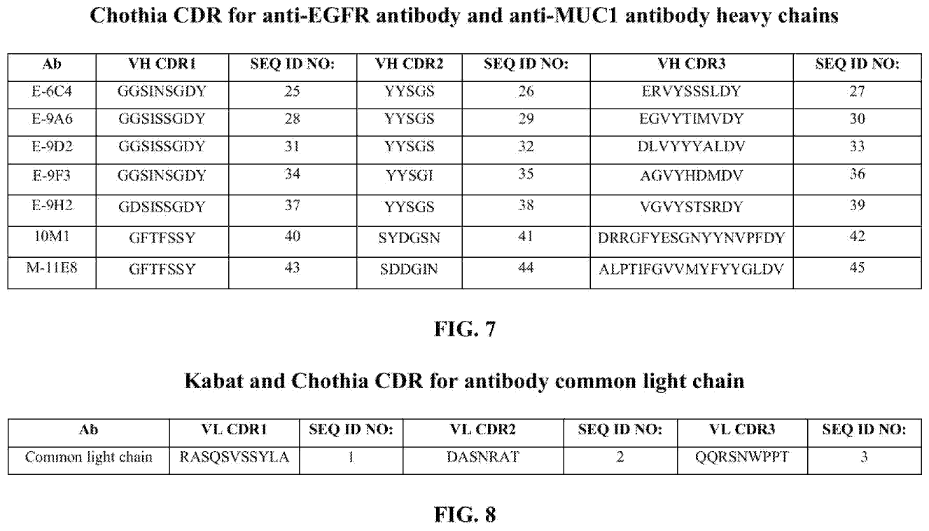

In some embodiments, the anti-EGFR/MUC1 antibodies or antigen-binding fragments thereof can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 6-8.

In some embodiments, the anti-EGFR/MUC1 antibody or antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the anti-EGFR/MUC1 antibody or antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the anti-EGFR/MUC1 antibody or antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence.

In some embodiments, the anti-EGFR/MUC1 antibodies contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 47, 48, 49, 50, 51, 52, or 53, and the selected VL sequence is SEQ ID NO: 46.

In some embodiments, the anti-EGFR/MUC1 antibody or antigen-binding fragment can have 3 VH CDRs that are identical to the CDRs of any VH sequences as described herein. In some embodiments, the anti-EGFR/MUC1 antibody or antigen-binding fragment can have 3 VL CDRs that are identical to the CDRs of any VL sequences as described herein.

The disclosure also provides nucleic acid comprising a polynucleotide encoding an anti-EGFR/MUC1 antibody. The immunoglobulin heavy chain or immunoglobulin light chain in the anti-EGFR/MUC1 antibody comprises CDRs as shown in FIGS. 6-8. When the polypeptides are paired with corresponding polypeptide (e.g., a corresponding heavy chain variable region or a corresponding light chain variable region), the paired polypeptides bind to MUC1 and/or EGFR.

The anti-EGFR/MUC1 antibodies can also be anti-EGFR/MUC1 antibody variants (including derivatives and conjugates) of anti-EGFR/MUC1 antibodies or antibody fragments.

Additional anti-EGFR/MUC1 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-EGFR/MUC1 antibodies can be of any type (e.g., IgG, IgE, IgM, IgD, IgA, and IgY), class (e.g., IgG1, IgG2, IgG3, IgG4, IgA1, and IgA2), or subclass. In some embodiments, the anti-EGFR/MUC 1 antibody or antigen-binding fragment is an IgG (e.g., IgG1) antibody or antigen-binding fragment thereof.

Fragments of anti-EGFR/MUC1 antibodies are suitable for use in the methods provided so long as they retain the desired affinity and specificity to both MUC1 and EGFR. Thus, a fragment of an anti-EGFR/MUC1 antibody will retain an ability to bind to MUC1 and EGFR.

Antibodies and Antigen Binding Fragments thereof. In some embodiments, the multispecific anti-EGFR/MUC1 antibody (e.g., bispecific antibody) includes an antigen-binding domain that is derived from an anti-EGFR antibody, and an antigen-binding domain that is derived from an anti-MUC1 antibody. These anti-EGFR/MUC1 antibodies and antigen-binding fragments thereof can have various forms.

In general, antibodies (also called immunoglobulins) can be made up of two classes of polypeptide chains, light chains and heavy chains. A non-limiting anti-EGFR/MUC1 antibody of the present disclosure can be an intact, four immunoglobulin chain antibody comprising two heavy chains and two light chains. The heavy chain of the anti-EGFR/MUC1 antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain.

The hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding domain.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (Oct. 1997); Morea et al., J Mol Biol. 275(2):269-94 (Jan 0.1998); Chothia et al., Nature 342(6252):877-83 (Dec. 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen-binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the anti-EGFR/MUC1 antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The anti-EGFR/MUC1 antibody can also be an immunoglobulin molecule that is derived from any species (e.g., human, rodent, mouse, rat, camelid). The antigen-binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an anti-EGFR/MUC1 antibody or antigen binding fragment thereof can comprise e.g., a scFv, a Fv, a Fd, a dAb, a bispecific antibody, a bispecific scFv, a diabody, a linear antibody, a single-chain antibody molecule, a multispecific antibody formed from antibody fragments, and any polypeptide that includes a binding domain which is, or is homologous to, an antibody binding domain. Non-limiting examples of antigen-binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv in an anti-EGFR/MUC1 antibody has two heavy chain variable domains, and two light chain variable domains. In some embodiments, the anti-EGFR/MUC1 scFv has two antigen binding regions (Antigen binding regions: A and B), and the two antigen binding regions can bind to the respective target antigens with different affinities.

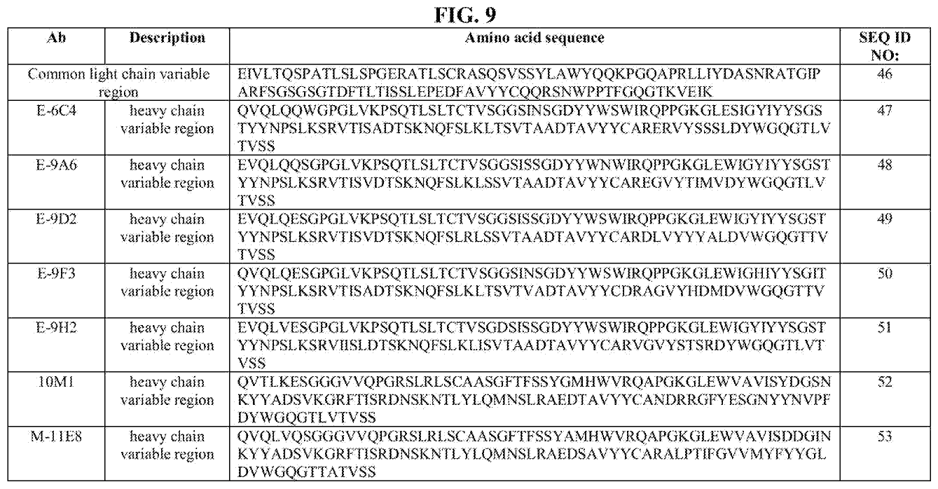

In some embodiments, the anti-EGFR/MUC1 antibodies or antigen-binding fragments thereof can comprises one, two, or three heavy chain variable region CDRs selected from FIGS. 6 and 7.

In some embodiments, the anti-EGFR/MUC1 antibodies described herein can be conjugated to a therapeutic agent. The anti-EGFR/MUC1 antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF. In some embodiments, the therapeutic agent is conjugated via a linker, e.g., a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

In some embodiments, the anti-EGFR/MUC1 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-EGFR/MUC1 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-EGFR/MUC1 antibody or an antigen-binding fragment in vitro (e.g., in tissue culture or when stored as a pharmaceutical composition) or in vivo (e.g., in a human).

The anti-EGFR/MUC1 antibodies or antigen-binding fragments thereof can also have various forms. Many different formats of bispecific antibodies or antigen-binding fragments thereof are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the anti-EGFR/MUC1 antibody is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the anti-EGFR/MUC1 antibody is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a dx-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fe, a Diabody-Fe, a tandem scFv-Fc, an Intrabody, a dock and lock, a ImmTAC, an IgG-IgG conjugate, a Cov-X-Body, or a scFv1-PEG-scFv2.

In some embodiments, the anti-EGFR/MUC1 antibody can be a TrioMab. In a TrioMab, the two heavy chains are from different species, wherein different sequences restrict the heavy-light chain pairing.

In some embodiments, the anti-EGFR/MUC1 antibody has two different heavy chains and one common light chain. Heterodimerization of heavy chains can be based on the knobs-into-holes or some other heavy chain pairing technique.

In some embodiments, CrossMAb technique can be used produce bispecific anti-EGFR/MUC1 antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies.

These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

The anti-EGFR/MUC1 antibody can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the anti-EGFR/MUC1 antibody can have a IgG-IgG format. Two intact IgG antibodies are conjugated by chemically linking the C-terminals of the heavy chains.

The anti-EGFR/MUC1 antibody can also have a Fab-scFv-Fc format. In Fab-scFv-Fc format, a light chain, heavy chain and a third chain containing the Fe region and the scFv are assembled. It can ensure efficient manufacturing and purification.

In some embodiments, the anti-EGFR/MUC1 antibody can be a TF. Three Fab fragments are linked by disulfide bridges. Two fragments target the tumor associated antigen (TAA) and one fragment targets a hapten. The TF format does not have an Fc region.

ADAPTIR has two scFvs bound to each side of an Fc region. It abandons the intact IgG as a basis for its construct, but conserves the Fe region to extend the half-life and facilitate purification.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fe, an Fc region is attached to the DART structure. It can be generated by assembling three chains, two via a disulfide bond, as with the DART. One chain contains half of the Fc region which will dimerize with the third chain, only expressing the Fe region. The addition of Fc region enhances half-life leading to longer effective concentrations, avoiding continuous IV.

In tetravalent DART, four peptide chains are assembled. Basically, two DART molecules are created with half an Fe region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In some embodiments, the anti-EGFR/MUC1 antibody is a bispecific antibody. In some embodiments, the bispecific antibody in present disclosure is designed to be 1+1 (monovalent for each target) and has an IgG1 subtype structure. This can reduce the avidity to cells with low expression levels of EGFR and MUC1, and increase the avidity to cells that co-express EGFR and MUC1, to achieve enhanced targeting function.

In some embodiments, the anti-EGFR/MUC1 antibodies or antigen-binding fragments thereof have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 54, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 55 and 56.

In some embodiments, the anti-EGFR/MUC1 antibodies include KIH mutations. In some embodiments, the anti-EGFR/MUC1 antibody includes a first antigen-binding domain that specifically binds to EGFR, and a second antigen-binding domain that specifically binds to MUC1. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that including one or more hole mutations (a hole heavy chain), and the second antigen-binding domain includes a heavy chain including one or more knob mutations (a knob heavy chain). In some embodiments, the anti-EGFR/MUC1 antibody includes a knob heavy chain comprising a constant region that is at least 80%, 85%, 86%, 87%, 88%, 89%, 90%, 91%, 92%, 93% 94%, 95%, 96%, 97%, 98%, 99% or 100% identical to SEQ ID NO: 55. In some embodiments, the anti-EGFR/MUC 1 antibody includes a hole heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 56.

### Antibody Characteristics

The anti-EGFR/MUC1 antibodies can include any anti-EGFR antigen-binding domain and any anti-MUC1 antigen-binding domain as described herein.

The disclosure provides anti-EGFR/MUC1 antibodies and antigen-binding fragments thereof that can specifically bind to EGFR. These anti-EGFR/MUC1 antibodies can be agonists or antagonists. The anti-EGFR/MUC1 antibodies, or antigen-binding fragments thereof described herein can bind to EGFR, and block the binding between EGFR and its ligands. By blocking the binding between EGFR and its ligands, the anti-EGFR/MUC1 antibodies can inhibit the EGFR-associated signaling pathway and thus treating cancer. In some embodiments, the anti-EGFR/MUC1 antibodies, or antigen-binding fragments thereof can initiate CMC or ADCC.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the anti-EGFR/MUC1 antibodies or antigen-binding fragments thereof can bind to EGFR (e.g., human EGFR, monkey EGFR, mouse EGFR, and/or chimeric EGFR) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s−¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

In some embodiments, the anti-EGFR/MUC1 antibodies or antigen-binding fragments thereof can bind to EGFR (e.g., human EGFR, monkey EGFR, mouse EGFR, and/or chimeric EGFR) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁵ M, greater than 1×10⁻⁹ M, or greater than 1×10⁻¹⁰ M.

The anti-EGFR/MUC1 antibodies or antigen-binding fragments thereof can also include an antigen-binding domain that can specifically bind to MUC1. The anti-EGFR/MUC1 antibodies or antigen-binding fragments thereof described herein can block the binding between MUC1 and its ligands. In some embodiments, by binding to MUC1, the anti-EGFR/MUC1 antibody can also inhibit MUC1-associated signaling pathways, thereby inhibiting cell proliferation, differentiation, and/or metastasis. Thus, in some embodiments, the anti-EGFR/MUC1 antibodies as described herein are MUC1 agonist. In some embodiments, the anti-EGFR/MUC1 antibodies are MUC1 antagonist.

In some implementations, the anti-EGFR/MUC1 antibodies or antigen-binding fragments thereof can bind to MUC1 (e.g., human MUC1, monkey MUC1, mouse MUC1, and/or chimeric MUC1) with a dissociation rate (koff) of less than 0.1 s¹, less than 0.01 s¹, less than 0.001 s¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon).

In some embodiments, KD is less than 1×10⁻⁶M, less than 1×10⁻⁷M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

Because the anti-EGFR/MUC1 antibody (e.g., bispecific antibody) binds to both MUC1 and EGFR, for cells that express both MUC1 and EGFR, the antibody has a higher binding affinity to these cells. Avidity can be used to measure the binding affinity of an antibody to these cells. Avidity is the accumulated strength of multiple affinities of individual non-covalent binding interactions.

Thermal stabilities can also be determined. The anti-EGFR/MUC1 antibodies or antigen-binding fragments thereof as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 T. As IgG can be described as a multi-domain protein, the melting curve sometimes shows two transitions, with a first denaturation temperature, Tm D 1, and a second denaturation temperature Tm D2. The presence of these two peaks often indicate the denaturation of the Fc domains (Tm D1) and Fab domains (Tm D2), respectively. When there are two peaks, Tm usually refers to Tm D2. Thus, in some embodiments, the anti-EGFR/MUC 1 antibodies or antigen-binding fragments thereof as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the anti-EGFR/MUC1 antibodies or antigen-binding fragments thereof as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm Dt, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the anti-EGFR/MUC1 antibodies or antigen-binding fragments thereof can bind to human EGFR or monkey EGFR. In some embodiments, the anti-EGFR/MUC1 antibodies or antigen-binding fragments thereof cannot bind to human EGFR or monkey EGFR. In some embodiments, the anti-EGFR/MUC1 antibodies or antigen-binding fragments thereof can bind to human MUC1 or monkey MUC1. In some embodiments, the anti-EGFR/MUC1 antibodies or antigen-binding fragments thereof cannot bind to human MUC1 or monkey MUC1.

In some embodiments, the anti-EGFR/MUC1 antibody, antigen-binding fragment, or ADC has a purity that is greater than 30%, 40%, 50%, 60%, 70%, 72.5%, 75%, 77.5%, 80%, 82.5%, 85%, 87.5%, 90%, 91% 92%, 93%, 94%, 95%, 96%, 97%, 98%, or 99%, e.g., as measured by HPLC. In some embodiments, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC.

In some embodiments, the anti-EGFR/MUC1 antibody, antigen-binding fragment, or ADC has a purity of above 90%, above 91%, above 92%, above 93%, above 94%, above 95%, above 96%, above 97%, or above 98%, as determined by size exclusion chromatography (SEC).

In some embodiments, the anti-EGFR/MUC1 antibody, antigen-binding fragment, or ADC has a hydrophobic interaction chromatography (HIC) retention time that is longer than 2 minutes, longer than 3 minutes, longer than 4 minutes, or longer than 5 minutes. In some embodiments, the HIC retention time is less than 2 minutes, less than 3 minutes, less than 4 minutes, less than 5 minutes, or less than 6 minutes.

In some embodiments, the anti-EGFR/MUC1 antibody, antigen-binding fragment, or ADC has a purity of above 85%, above 86%, above 87%, above 88%, above 89%, above 90%, above 91%, above 92%, above 93%, above 94%, above 95%, above 96%, above 97%, above 98%, or above 99%, as determined by capillary electrophoresis-sodium dodecyl sulphate (CE-SDS).

In some embodiments, the anti-EGFR/MUC1 antibody, antigen-binding fragment, or ADC has a main peak that constitutes more than 40%, more than 50%, more than 55%, more than 60%, more than 65%, more than 70%, more than 75%, more than 80%, more than 85%, or more than 90% of the total sample, as determined by capillary isoelectric focusing (cIEF). In some embodiments, the anti-EGFR/MUC1 antibody, antigen-binding fragment, or ADC has an acidic peak that constitutes less than 10%, less than 15%, less than 20%, less than 25%, less than 30%, less than 35%, less than 40%, less than 45%, less than 50%, less than 55%, less than 60%, less than 65%, less than 70%, or less than 75% of the total sample, as determined by capillary isoelectric focusing (cIEF).

In some embodiments, the anti-EGFR/MUC1 antibody, antigen-binding fragment, or ADC has a tumor growth inhibition rate or percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the anti-EGFR/MUC1 antibody, antigen-binding fragment, or ADC has a tumor growth inhibition percentage that is less than 60%, 70%, 80%, 90%, 100%, 110%, 120%, 130%, 140%, or 150%. The TGI (%) can be determined, e.g., at 3, 4, 5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 days after the treatment starts. As used herein, the tumor growth inhibition rate or percentage (TGI %) is calculated using the following formula:

$$TGI ( \% ) = [ 1 - ( Ti - T 0 ) / ( Vi ‐ V 0 ) ] \times 100 \%$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the anti-EGFR/MUC1 antibody, antigen-binding fragment, or ADC has a functional Fe region. In some embodiments, effector function of a functional Fe region is antibody-dependent cell-mediated cytotoxicity (ADCC). In some embodiments, effector function of a functional Fc region is phagocytosis. In some embodiments, effector function of a functional Fe region is ADCC and phagocytosis. In some embodiments, the Fe region is human IgG1, human IgG2, human IgG3, or human IgG4.

In some embodiments, the anti-EGFR/MUC1 antibody, antigen-binding fragment, or ADC does not have a functional Fe region. For example, the anti-EGFR/MUC1 antibodies or antigen-binding fragments thereof are Fab, Fab′, F(ab′)₂, and Fv fragments. In some embodiments, the anti-EGFR/MUC1 antibodies or antigen-binding fragments thereof as described herein have an Fc region without effector function. In some embodiments, the Fc is a human IgG4 Fe. In some embodiments, the Fe does not have a functional Fe region. For example, the Fc region has LALA mutations (L234A and L235A mutations in EU numbering), or LALA-PG mutations (L234A, L235A, P329G mutations in EU numbering).

Some other modifications to the Fe region can be made. For example, a cysteine residue(s) can be introduced into the Fe region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fe region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fe region (Eu numbering of Fc region residues; or position 314 in Kabat numbering); however, Asn297 may also be located about:3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fe region can be further engineered to replace the Asparagine at position 297 with Alanine (N297A).

In some embodiments, the main peak of HPLC-SEC accounts for at least 70%, 75%, 80%, 85%, 90%, 95%, 96%, 97%, 98%, 99%, 99.5%, 100% of the protein complex described herein after purification by protein A-based affinity chromatography and/or size-exclusive chromatography.

In some embodiments, the anti-EGFR/MUC1 ADC described herein has an IC50 for in vitro killing of cancer cells of less than 5 μg/mL, less than 4.5 μg/mL, less than 4 μg/mL, less than 3.5 μg/mL, less than 3 μg/mL, less than 2.5 μg/mL, less than 2 μg/mL, less than 1.5 μg/mL, less than 1 μg/mL, less than 0.9 μg/mL, less than 0.8 μg/mL, less than 0.7 μg/mL, less than 0.6 pg/mL, less than 0.5 μg/mL, less than 0.4 g/mL, less than 0.3 μg/mL, less than 0.2 g/mL, less than 0.1 μg/mL, less than 0.05 μg/mL, less than 0.025 μg/mL, less than 0.0125 μg/mL, less than 0.005 μg/mL, or less than 0.0025 μg/mL.

In some embodiments, the bispecific anti-EGFR/MUC1 antibody described herein has a higher endocytosis rate than the corresponding monoclonal antibodies and/or control antibodies described herein. In some embodiments, the anti-EGFR/MUC1 antibody described herein has a higher endocytosis rate than Cetuximab analog and/or Gatipotuzumab analog. In some embodiments, the endocytosis rate can be measured by the percentage of positive population. In some embodiments, the bispecific anti-EGFR/MUC1 antibody described herein has a percentage of positive population that is higher than 70%, higher than 75%, higher than 80%, higher than 85%, higher than 90%, higher than 95%, higher than 96%, higher than 97%, higher than 98%, higher than 99%, or higher than 99.5%.

In some embodiments, the bispecific anti-EGFR/MUC1 antibody described herein has a higher binding activity to cells (e.g., HCC827 cells, HCC70 cells, A431 cells, or ZR-75-1 cells) than the corresponding monoclonal antibodies and/or control bispecific antibodies described herein. In some embodiments, the binding activity to cells can be determined by EC50. In some embodiments, the bispecific anti-EGFR/MUC1 antibody described herein has an EC50 that is less than 30 μg/mL, less than 20 μg/mL, less than 10 μg/mL, less than 5 μg/mL, less than 4 μg/mL, less than 3 μg/mL, less than 2 μg/mL, less than 1 μg/mL, or less than 0.5 μg/mL.

### Antibody Drug Conjugates (ADC)

The anti-EGFR/MUC1 antibodies or antigen-binding fragments thereof described herein can be conjugated to a therapeutic agent (a drug). The therapeutic agent can be covalently or non-covalently bind to the anti-EGFR/MUC1 antibody. In some embodiments, the anti-EGFR/MUC1 antibody is an anti-EGFR/MUC1 bispecific antibody. In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

Definitions of specific functional groups and chemical terms are described in more detail below. For purpose of this invention, the chemical elements are identified in accordance with the Periodic Table of the Elements, CAS version, Handbook of Chemistry and Physics, 75^{th} Edition, inside cover, and specific functional groups are generally defined as described therein.

Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987.

All ranges cited herein are inclusive, unless expressly stated to the contrary. When a range of values is listed, it is intended to encompass each value and sub-range within the range.

For example, “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The compounds or any formula depicting and describing the compounds of the present disclosure may have one or more chiral (asymmetric) centers. The present invention encompasses all stereoisomeric forms of the compounds or any formula depicting and describing the compounds of the present invention. Centers of asymmetry that are present in the compounds or any formula depicting and describing the compounds of the present invention can all independently of one another have (R) or (S) configuration. When bonds to a chiral carbon are depicted as straight lines in the structural formulas, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the formula or by the name.

The disclosure includes all possible enantiomers and diastereomers and mixtures of two or more stereoisomers, for example mixtures of enantiomers and/or diastereomers, in all ratios.

Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism the disclosure includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis.

Optionally a derivatization can be carried out before a separation of stereoisomers. The separation of a mixture of stereoisomers can be carried out at an intermediate step during the synthesis of a compound or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds or any formula depicting and describing the compounds of the present invention.

Examples of isotopes suitable for inclusion in the compounds of the present invention include, but not limited to, isotopes of hydrogen, such as ²H (i.e., D) and ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S Certain isotopic variants of the compounds or any formula depicting and describing the compounds of the present disclosure, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (²H, or D), can afford certain therapeutic advantages, for example, resulting from greater metabolic stability, increased in vivo half-life, or reduced dosage requirements and, hence, may be utilized in some particular circumstances. Isotopic variants of compounds or any formula depicting and describing the compounds of the present disclosure can generally be prepared by techniques known to those skilled in the art or by processes analogous to those described in the accompanying examples and synthesis using an appropriate isotopically-labeled reagent in place of the non-labeled reagent previously employed.

The compounds as provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present disclosure contain one or more acidic or basic groups, the disclosure also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present disclosure include but are not limited to copper (I), copper (II), iron (II), iron (III), manganese (II), and zinc salts. Compounds of the present disclosure which contain one or more basic groups, e.g., groups which can be protonated, can be present in salt form, and can be used according to the disclosure in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the disclosure may moreover be an integral or non-integral multiple of one.

Compounds of the present disclosure which contain basic nitrogen-containing groups can be quaternized using agents such as C₁₋₄alkyl halides, for example, methyl, ethyl, isopropyl, and tert-butyl chloride, bromide, and iodide; diC₁₋₄alkyl sulfates, for example, dimethyl, diethyl, and diamyl sulfate; C₁₀₋₁₈alkyl halides, for example, decyl, dodecyl, lauryl, myristyl, and stearyl chloride, bromide, and iodide; and arylC₁₋₄ alkyl halides, for example, benzyl chloride and phenethyl bromide.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound or any formula depicting and describing the compounds of the present disclosure and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present disclosure may include those wherein the solvent of crystallization may be isotopically substituted, e.g., D₂O, d₆-acetone, d₆-DMSO.

### Linker (Linking Agent Compound)

In some embodiments, the therapeutic agent is conjugated via a linker (or a linking agent compound). As used herein, the term “linker” or “linking agent compound” refers to a compound that can connect a ligand (e.g., the antibodies or the antigen-binding fragments thereof described herein) and a therapeutic agent (e.g., any of the therapeutic agents described herein) together to form a ligand-drug conjugate by reacting with a group of the ligand compound and the therapeutic agent compound respectively by, for example, a coupling reaction.

In some embodiments, the linker described herein is a compound having the following formula:

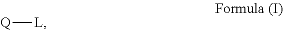

or a pharmaceutically acceptable salt, solvate, stereoisomer, or isotopic variant thereof, wherein Q denotes to a junction moiety capable of being coupled to a ligand via a bond selected from the group consisting of carbonyl, thioether, amide, disulfide and hydrazone bond; L denotes to a linker moiety capable of connecting Q to a therapeutic agent.

In some embodiments, the junction moiety (Q in Formula (I)) has the following structure:

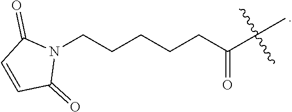 [SMILES: *C(=O)CCCCCN1C(=O)C=CC1=O]

In some embodiments, the linker moiety (L in Formula (1)) has the following formula:

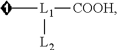

where L₁ is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;

L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ,(CH₂)ₚC(O)OH, and —(CH₂O) (CH₂CH₂O)ₘ(CH₂)ₚC(O)NHR^{L2d}, R^{L2a} is H or C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and denotes to the N-terminal side of the polypeptide residue covalently attached to the junction moiety Q.

In some embodiments, the polypeptide residue L_{T} is ^{NH}-Glu-Val-Ala-^{COOH} In some embodiments, the hydrophilic group L₂has the following structure:

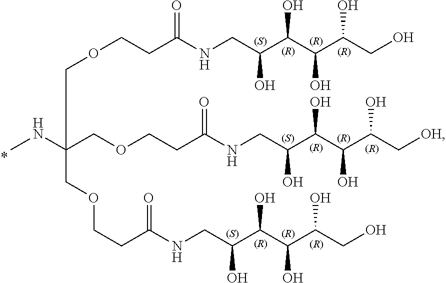

wherein “*” denotes the site covalently attached to polypeptide residue L₁, e.g., side chain of the Glu residue in ^{NH}-Glu-Val-Ala-^{COOH}

In some embodiments, the linker described herein is a compound having the following structure:

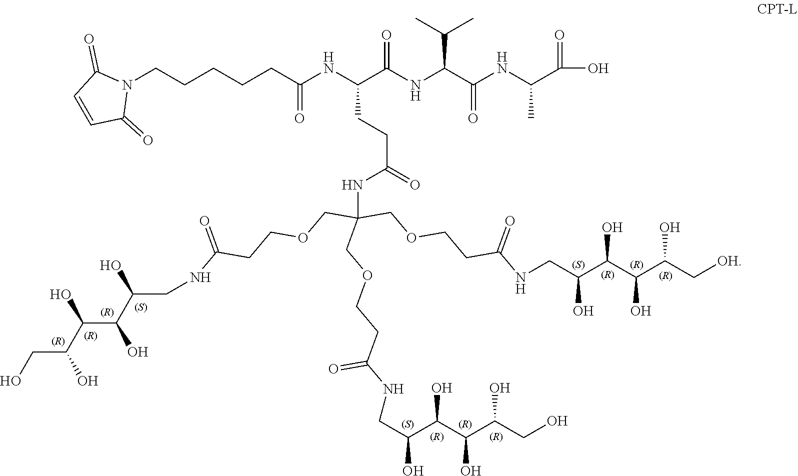

In some embodiments, the linker is a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

### Therapeutic Agent

In some embodiments, the therapeutic agent that is conjugated to the antibodies or the antigen-binding fragments thereof described herein is discussed as follows.

In some embodiments, the therapeutic agent described herein is a cytotoxic agent. In some embodiments, the cytotoxic agent is a camptothecin compound, an analogue or a derivative thereof. In some preferred embodiments, the camptothecin compound is a compound having the following structure:

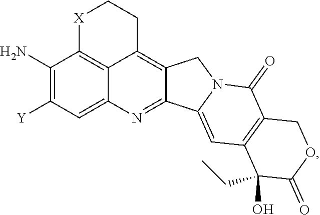

wherein X is selected from the group consisting of —CH2—, O and S; Y is selected from the group consisting of H, D, and F.

In some embodiments, the therapeutic agent is (S)-4-amino-9-ethyl-9-hydroxy-1,9,12, 15-tetrahydro-13H-pyrano[3′,4′:6,7]indolizino[1,2-b]thiopyrano[4,3,2-de]quinoline-10,13(2H)-di one) (CPT-1). The structure of CPT-1 is shown below:

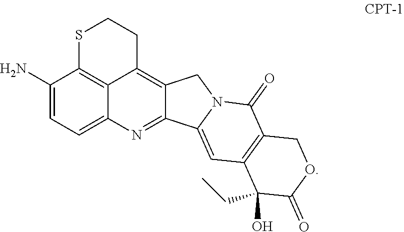 [SMILES: CC[C@@]1(O)C(=O)OCc2c1cc1n(c2=O)Cc2c-1nc1ccc(N)c3c1c2CCS3]

In some embodiments, the therapeutic agent is (S)-4-amino-9-ethyl-9-hydroxy-1,9,12,15-tetrahydro-13H-pyrano[4,3,2-de]pyrano[3′,4′:6,7]indolizino[1,2-b]quinoline-10,13(2H)-dione (CPT-2). The structure of CPT-2 is shown below:

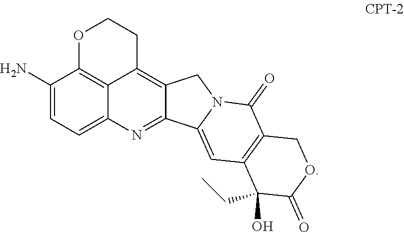 [SMILES: CC[C@@]1(O)C(=O)OCc2c1cc1n(c2=O)Cc2c-1nc1ccc(N)c3c1c2CCO3]

In some embodiments, the therapeutic agent is CPT3. The structure of CPT-3 is shown

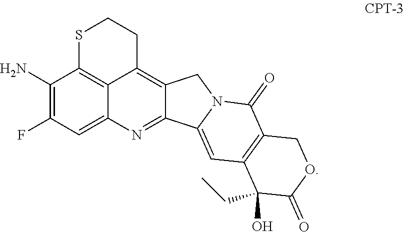 [SMILES: CC[C@@]1(O)C(=O)OCc2c1cc1n(c2=O)Cc2c-1nc1cc(F)c(N)c3c1c2CCS3]

In some embodiments, the therapeutic agent is (S)-4-amino-9-ethyl-5-fluoro-9-hydroxy-1,9,12,15-tetrahydro-13H-pyrano[4,3,2-de]pyrano[3′,4′:6,7]indolizino[1,2-b]quinoline-10,13(2H)-dione (CPT-4). The structure of CPT-4 is shown below:

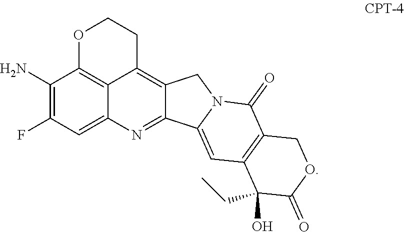 [SMILES: CC[C@@]1(O)C(=O)OCc2c1cc1n(c2=O)Cc2c-1nc1cc(F)c(N)c3c1c2CCO3]

In some embodiments, the therapeutic agent is an auristatin, such as auristatin E (also known in the art as a derivative of dolastatin-10) or a derivative thereof. The auristatin can be, is for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

### Linker-Therapeutic Agent Compound

In some embodiments, a linker (e.g., any of the linkers described herein) and a therapeutic agent (e.g., any of the therapeutic agents described herein) can be linked to form a “linker-therapeutic agent” compound.

In some embodiments, the linker-therapeutic agent compound has the following structure:

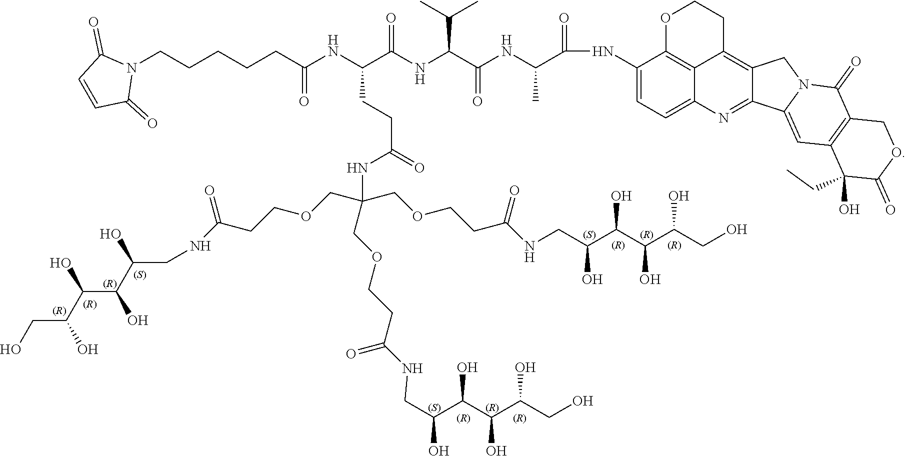

In some embodiments, the linker-therapeutic agent compound has the following structure:

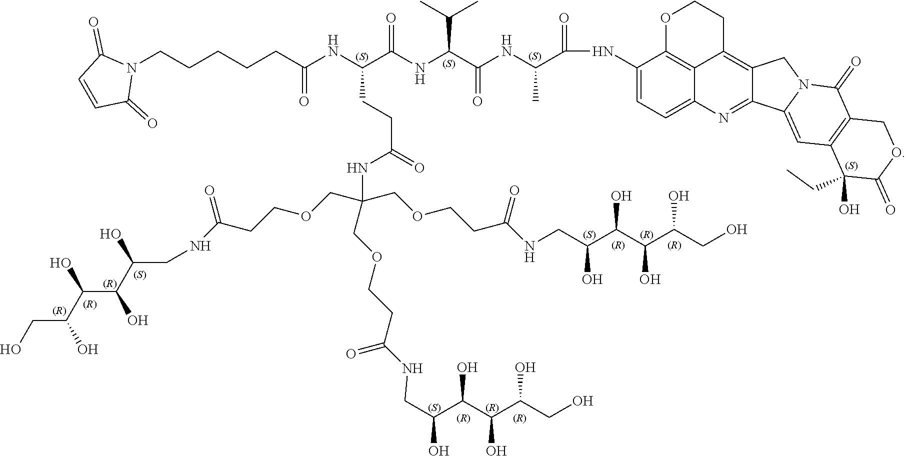

In some embodiments, an antibody (“Ab”), e.g., any of the antibodies or the antigen-binding fragments thereof described herein, can be linked to a linker-therapeutic agent compound (e.g., any of the linker-therapeutic agent compounds described herein) to generate an antibody-drug conjugate. In some embodiments, the antibody-drug conjugate has the following structure:

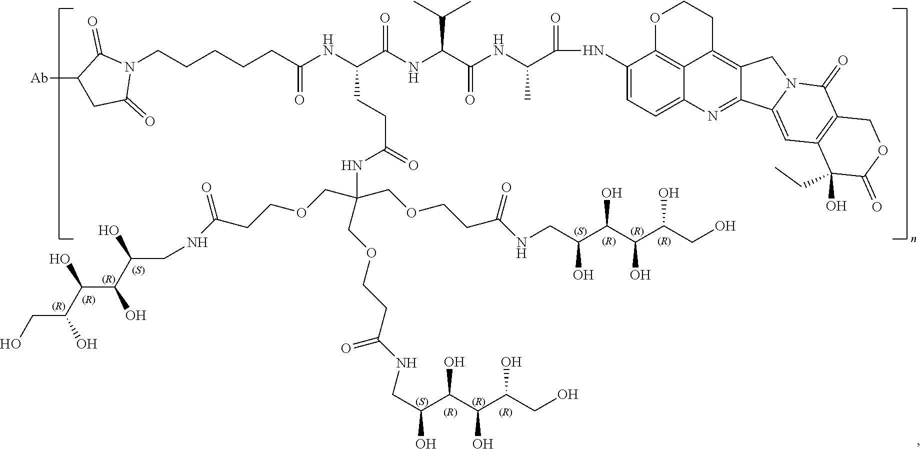

wherein n=1-8. In some embodiments, n=1-8. In some embodiments, n is about 1, about 2, about 3, about 4, about 5, about 6, about 7, about 8. In some embodiments, n is about 1-8, 1-7, 1-6, 1-5, 1-4, 1-3, 1-2, 2-8, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8. In some embodiments, n is an integral or non-integral multiple of one.

In some embodiments, the anti-EGFR/MUC1 antibody is coupled to the drug via a cleavable linker e.g. a SPBD linker or a maleimidocaproyl-valine-citrulline-p-aminobenz yloxycarbonyl (VC) linker.

In some embodiments, the anti-EGFR/MUC1 antibody is coupled to the drug via a non-cleavable linker e.g. a MCC linker formed using SMCC or sulfo-SMCC. Selection of an appropriate linker for a given ADC can be readily made by the skilled person hav ing knowledge of the art and taking into account relevant factors, such as the site of att achment to the anti-EGFR/MUC1 antibody, any structural constraints of the drug and the hydrophobicity of the drug (see, for example, review in Nolting, Chapter 5, Antibody-Dr ug Conjugates: Methods in Molecular Biology, 2013, Ducry (Ed.), Springer). A number o f specific linker-toxin combinations have been described and may be used with the anti-EGFR/MUC1 antibodies or antigen-binding fragments thereof described herein to prepare ADCs in certain embodiments. Examples include, but are not limited to, cleavable peptid e-based linkers with auristatins such as MMAE and MMAF, camptothecins such as SN-3 8, duocarmycins and PBD dimers; non-cleavable MC-based linkers with auristatins MMA F and MMAE; acid-labile hydrazone-based linkers with calicheamicins and doxorubicin; d isulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Pu blication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the anti-EGFR/MUC1 antibodies or antigen-binding fragments thereof through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The anti-EGFR/MUC1 ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the anti-EGFR/MUC1 ADCs described here. Various prepared linkers, linker components and toxins are commercially available or may be prepared using standard synthetic organic chemistry techniques. These methods are described e.g., in March's Advanced Organic Chemistry (Smith & March, 2006, Sixth Ed., Wiley); Toki et al., (2002) J. Org. Chem. 67:1866-1872; Frisch et al., (1997) Bioconj.

Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected anti-EGFR/MUC1 antibody or antigen-binding fragment are also available commercially, for example, linker-toxins comprising DM1, DM4, MMAE, MMAF or Duocarmycin SA are available from Creative BioLabs (Shirley, N.Y.).

Several specific examples of methods of preparing anti-EGFR/MUC1 ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p>5, may cause aggregation, insolubility, toxicity, or loss of cellular permeability of certain antibody-drug conjugates. In certain embodiments, the average drug loading for an anti-EGFR/MUC1 antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) for an anti-EGFR/MUC 1 ADC composition is about or at least 1, 2, 3, 4, 5, 6, 7, or 8. In some embodiments, the average DAR in the anti-EGFR/MUC1 ADC composition is about 1~about 2, about 2-about 3, about 3-about 4, about 3-about 5, about 4-about 5, about 5-about 6, about 6-about 7, or about 7-about 8.

In some embodiments, anti-EGFR/MUC1 antibody variants have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fe region (Eu numbering of Fe region residues; or position 314 in Kabat numbering); however, Asn297 may also be located about:3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the anti-EGFR/MUC1 antibody can be further engineered to replace the Asparagine at position 297 with Alanine (N297A).

In some embodiments, to facilitate production efficiency by avoiding Fab-arm exchange, the Fc region of the anti-EGFR/MUC1 antibodies or antigen-binding fragments thereof was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific anti-EGFR/MUC1 antibody. Bispecific anti-EGFR/MUC1 antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knobs-into-holes (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7.

No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (FU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of anti-EGFR/MUC1 antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. co/i and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an anti-EGFR/MUC1 antibody of the present disclosure by higher eukaryotes may be increased by inserting an enhancer sequence into the vector.

Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., an anti-EGFR/MUC1 antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide.

The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to EGFR. In some embodiments, a pair of vectors is provided, wherein each vector comprises one of the nucleic acids as described herein, wherein together the pair of vectors encodes the VL region and the VH region that together bind to EGFR.

In some embodiments, the vector includes two of the nucleic acids as described herein, wherein the vector encodes the VL region and the VH region that together bind to MUC1. In some embodiments, a pair of vectors is provided, wherein each vector comprises one of the nucleic acids as described herein, wherein together the pair of vectors encodes the VL region and the VH region that together bind to MUC1.

### Methods of Treatment

The methods described herein include methods for the treatment of disorders associated with cancer. Generally, the methods include administering a therapeutically effective amount of anti-EGFR/MUC1 antibodies or anti-EGFR/MUC1 antibody-drug conjugates as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth.

The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of anti-EGFR/MUC1 antibodies or an anti-EGFR/MUC1 antibody drug conjugates disclosed herein to a subject in need thereof, e.g., a subject having, or identified or diagnosed as having, a cancer, e.g., solid tumor, lung cancer (e.g., non-small cell lung cancer, lung adenocarcinoma, or lung carcinoma), gastric cancer (e.g., gastric carcinoma), skin cancer (e.g., skin carcinoma), colorectal cancer, breast cancer, head and neck cancer, ovarian cancer, prostate cancer, thyroid cancer, pancreatic cancer, CNS cancer, liver cancer, nasopharynx cancer, brain cancer, colon cancer, bladder cancer, oral squamous cell carcinoma, cervical cancer, or oesophageal cancer. In some embodiments, the cancer is lung cancer, head and neck cancer, colon cancer, pancreatic cancer, breast cancer, ovarian cancer, bladder cancer, kidney cancer, gliomas, oesophageal cancer, gastric cancer, stomach cancer, prostate cancer, cervical cancer, multiple myeloma, or Non-Hodgkin Lymphoma.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the anti-EGFR/MUC1 antibody, anti-EGFR/MUC1 antigen binding fragment, anti-EGFR/MUC1 antibody-drug conjugates, anti-EGFR/MUC1 antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an anti-EGFR/MUC1 antibody, an anti-EGFR/MUC1 antigen binding fragment, or an anti-EGFR/MUC1 antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an anti-EGFR/MUC1 antibody, anti-EGFR/MUC1 antigen binding fragment, or anti-EGFR/MUC1 antibody-drug conjugate may vary, depending on, inter alia, patient history as well as other factors such as the type (and/or dosage) of the agent used.

Effective amounts and schedules for administering the anti-EGFR/MUC1 antibodies, anti-EGFR/MUC1 antigen-binding fragments thereof, anti-EGFR/MUC1 antibody-encoding polynucleotides, anti-EGFR/MUC1 antibody-drug conjugates, and/or compositions disclosed herein may be determined empirically, and making such determinations is within the skill in the art. Those skilled in the art will understand that the dosage that must be administered will vary depending on, for example, the mammal that will receive the anti-EGFR/MUC1 antibodies, anti-EGFR/MUC1 antigen-binding fragments thereof, anti-EGFR/MUC1 antibody-encoding polynucleotides, anti-EGFR/MUC1 antibody-drug conjugates, and/or compositions disclosed herein, the route of administration, the particular type of the agent or compositions disclosed herein used and other drugs being administered to the mammal.

A typical daily dosage of an effective amount of an anti-EGFR/MUC1 antibody or anti-EGFR/MUC1 ADC is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one anti-EGFR/MUC1 antibody, the anti-EGFR/MUC1 antigen-binding fragment thereof, anti-EGFR/MUC1 antibody-drug conjugates, or pharmaceutical composition (e.g., comprising any of the anti-EGFR/MUC1 antibodies, anti-EGFR/MUC1 antigen-binding antibody fragments, or anti-EGFR/MUC1 ADC) and, optionally, at least one additional therapeutic agent can be administered to the subject (e.g., once a week, twice a week, three times a week, four times a week, once a day, twice a day, or three times a day).

In some embodiments, the one or more additional therapeutic agents can be administered to the subject prior to, or after administering the at least one anti-EGFR/MUC1 antibody, anti-EGFR/MUC1 antigen-binding antibody fragment, anti-EGFR/MUC1 antibody-drug conjugate, or pharmaceutical composition (e.g., comprising any of the anti-EGFR/MUC1 antibodies, anti-EGFR/MUC1 antigen-binding antibody fragments, or anti-EGFR/MUC1 ADC). In some embodiments, the one or more additional therapeutic agents and the at least one anti-EGFR/MUC1 antibody, anti-EGFR/MUC1 antigen-binding antibody fragment, or anti-EGFR/MUC1 antibody-drug conjugate are administered to the subject such that there is an overlap in the bioactive period of the one or more additional therapeutic agents and the at least one anti-EGFR/MUC1 antibody, anti-EGFR/MUC1 antigen-binding fragment, or anti-EGFR/MUC1 ADC in the subject.

In some embodiments, the subject can be administered the at least one anti-EGFR/MUC1 antibody, anti-EGFR/MUC1 antigen-binding antibody fragment, anti-EGFR/MUC1 antibody-drug conjugate, or pharmaceutical composition (e.g., comprising any of the anti-EGFR/MUC1 antibodies, anti-EGFR/MUC1 antigen-binding antibody fragments, or anti-EGFR/MUC1 ADC) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of anti-EGFR/MUC1 antibodies or anti-EGFR/MUC1 antigen-binding antibody fragments, anti-EGFR/MUC1 antibody-drug conjugates (and/or one or more additional therapeutic agents) administered to the subject and can also adjust (e.g., increase or decrease) the dosage or frequency of administration of at least one anti-EGFR/MUC 1 antibody, anti-EGFR/MUC1 antigen-binding antibody fragment, or anti-EGFR/MUC1 ADC (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of MUC1, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDOl) (e.g., epacadostat).

In some embodiments, the additional therapeutic agent can comprise one or more inhibitors selected from the group consisting of an inhibitor of EGFR, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L 1 antibody, anti-PD-L2 antibody, an anti-LAG-3 antibody, an anti-TIGIT antibody, an anti-BTLA antibody, an anti-CTLA4 antibody, an anti-CD40 antibody, an anti-OX40 antibody, an anti-4-1BB antibody, an anti-TIM3 antibody, or an anti-GITR antibody.

### Pharmaceutical Compositions and Routes of Administration

Also provided herein are pharmaceutical compositions that contain at least one (e.g., one, two, three, or four) of the anti-EGFR/MUC1 antibodies (e.g., bispecific antibodies), anti-EGFR/MUC1 antigen-binding fragments, or anti-EGFR/MUC1 antibody-drug conjugates described herein. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the anti-EGFR/MUC1 antibody, anti-EGFR/MUC1 antigen-binding fragment thereof, or the anti-EGFR/MUC1 ADC can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the anti-EGFR/MUC1 antibodies, anti-EGFR/MUC1 antigen-binding fragments, anti-EGFR/MUC1 antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the anti-EGFR/MUC1 antibodies, an anti-EGFR/MUC1 antigen-binding fragment thereof, or an anti-EGFR/MUC1 ADC will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the anti-EGFR/MUC1 antibodies, the anti-EGFR/MUC1 antigen-binding fragment thereof, or the anti-EGFR/MUC1 ADC described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the anti-EGFR/MUC1 antibodies, the anti-EGFR/MUC1 antigen-binding fragment thereof, or the anti-EGFR/MUC1 ADC described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). While these doses cover a broad range, one of ordinary skill in the art will understand that therapeutic agents vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the therapeutic agent in vivo.

The pharmaceutical compositions can be included in a container, pack, or dispenser together with instructions for administration. The disclosure also provides methods of manufacturing the anti-EGFR/MUC1 antibodies, the anti-EGFR/MUC1 antigen-binding fragment thereof, or the anti-EGFR/MUC1 ADC for various uses as described herein.

### EXAMPLES

The invention is further described in the following examples, which do not limit the scope of the invention described in the claims.

### Example 1. Preparation and Analysis of Anti-EGFR/MUC1 Bispecific Antibodies

### Preparation of Anti-EGFR/MUC1 Bispecific Antibodies

Anti-EGFR antibodies (E-6C4, E-9A6, E-9D2, E-9F3 and E-9H2) and anti-MUC1 antibodies (10M1 and M-11E8) were paired to form various anti-EGFR/MUC1 bispecific antibodies. Specifically, vectors encoding the light chain and heavy chain of the antibodies were constructed. CHO-S cells were co-transfected with three vectors, including a first vector encoding the heavy chain of an anti-EGFR antibody, a second vector encoding the heavy chain of an anti-MUC1 antibody, and a third vector encoding the common light chain. After 14 days of culture, the cell supernatant was collected and purified by Protein A affinity chromatography.

Various methods can be used to reduce the chance of wrong pairing between the two heavy chains. In this example, knobs-into-holes mutations were introduced in the Fe regions of the anti-EGFR arm heavy chain and the anti-MUC1 arm heavy chain. Exemplary bispecific antibodies obtained included: E-6C4-10M1, E-9A6-10M1, E-9D2-10M1, E-9F3-10M1, E-9H2-1OM1, E-6C4-M-11E8, E-9A6-M-11E8, E-9D2-M-11E8, E-9F3-M-11E8 and E-9H2-M-11E8.

To verify the binding affinity of bispecific antibodies, anti-EGFR or anti-MUC1 control bispecific antibodies were also generated, in which one arm of the control bispecific antibody recognizes EGFR or MUC1, and the other arm recognizes CD28. Similar methods (e.g., obtaining VH sequences by immunizing RenLite™ mice) were used to generate these control bispecific antibodies. Exemplary control bispecific antibodies are named as E-6C4-CD28, E-9A6-CD28, E-9D2-CD28, E-9F3-CD28, E-9H2-CD28, CD28-E-6C4, CD28-E-9A6, CD28-E-9D2, CD28-E-9F3, CD28-E-9H2, 10M1-CD28, M-11E8-CD28, CD28-10M1 and CD28-10M1.

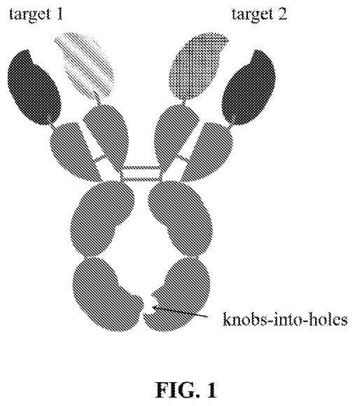

Knobs-into-holes mutations were introduced to all the bispecific antibodies. For example, in E-6C4-10M1, the heavy chain constant region of E-6C4 includes knob mutations, and the heavy chain constant region of 10M1 includes hole mutations. In E-6C4-CD28, the heavy chain constant region of E-6C4 includes knob mutations, and the heavy chain constant region of CD28 includes hole mutations. An exemplary antibody structure is shown in FIG. 1, where target 1 and target 2 can be (1) EGFR and MUC1; respectively; (2) MUC1 and EGFR, respectively; (3) EGFR and CD28, respectively; (4) CD28 and EGFR, respectively; (5) MUC1 and CD28, respectively; or (6) CD28 and MUC1, respectively.

The sequences of the light chain constant region, the heavy chain constant region with knob mutations, and the heavy chain constant region with hole mutations are shown in SEQ ID NO: 54, SEQ ID NO: 55 and SEQ ID NO: 56, respectively.

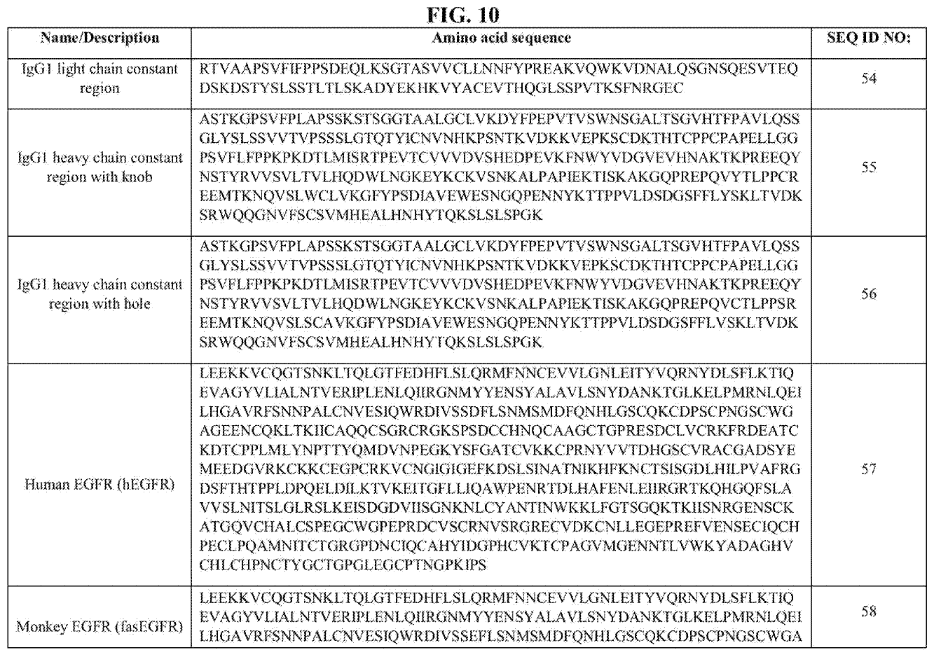

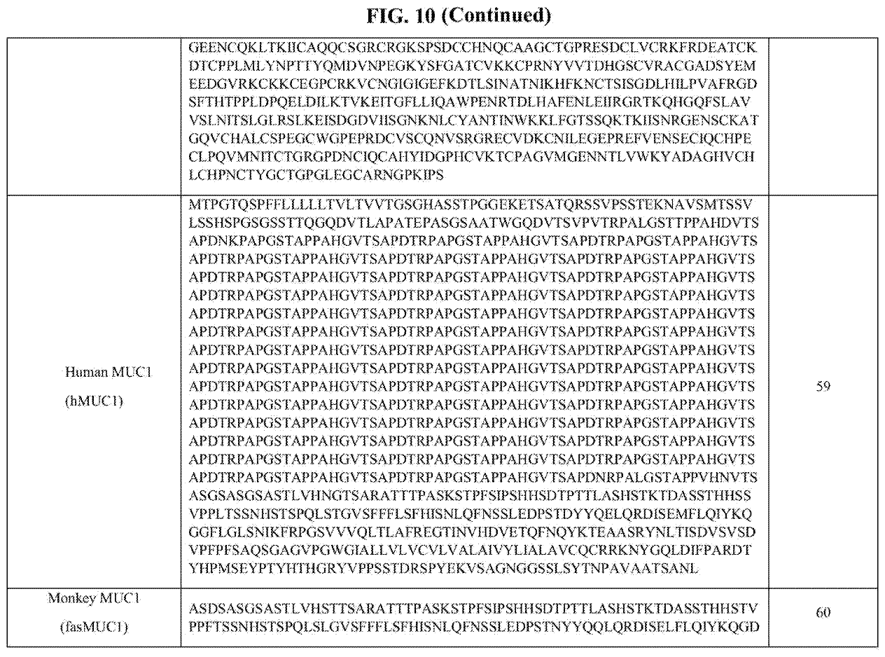

The VH and VL CDR sequences of E-6C4, E-9A6, E-9D2, E-9F3, E-9H2, 1OM1 and M-11 E8 are shown in FIG. 6, FIG. 7 and FIG. 8, and their VH and VL regions sequences are shown in FIG. 9.

### Cross-Species Binding of Anti-EGFR/MUC1 Bispecific Antibodies

CHO-S-hMUC1 (484AA) cells, CHO-S-fasMUC1 cells, CHO-S-hEGFR cells or CHO-S-fasEGFR cells were transferred to a 96-well plate at a density of 1×10⁵ cells/well. The anti-EGFR/MUC1 bispecific antibodies (5 μg/mL) were added to the 96-well plate, and incubated at 4° C. for 30 min. Then, the cells were incubated with the secondary antibody anti-hIgG-Fc-Alex Flour 647 (RLI-H) (Jackson ImmunoResearch Laboratories, Inc., Cat #: 109-606-170) at 4° C. in the dark for 15 minutes before flow cytometry analysis. For the negative control (NC), only cells were added and no antibodies were added.

CHO-S-hEGFR cells or CHO-S-fasEGFR cells were obtained by transfecting CHO-S cells with vectors expressing human EGFR (hEGFR, SEQ ID NO: 57) or monkey EGFR (fasEGFR, SEQ ID NO: 58) respectively. CHO-S-hMUC1 (484AA) cells were obtained by transfecting CHO cells with vectors expressing 484 amino acids of human MUC1 (hMUC1(484AA) (SEQ ID NO: 67). CHO-S-fasMUC1 cells were obtained by transfecting CHO-S cells with vectors expressing monkey (Macaca fascicularis) MUC1 (fasMUC1, SEQ ID NO: 60). For isotype control (ISO), an antibody targeting an irrelevant target protein was used.

The test results are shown in the table below. All anti-EGFR/MUC1 bispecific antibodies E-6C4-10M1, E-9A6-10M1, E-9D2-10M1, E-9F3-10M1, E-9H2-10M1, E-6C4-M-11E8,E-9A6-M-11E8, E-9D2-M-11E8, E-9F3-M-11E8 and E-9H2-M-llE8 can bind to human MUC1, monkey MUC1, human EGFR and monkey EGFR.

TABLE 1

| | | Percentage | |
|---|---|---|---|
| | | of Positive | |
| Antibody | Cells | Cells (%) | Evaluation |
| NC + secondary | CHO-S- | 2.00 | No Binding |
| antibody | hMUC1(484AA) | | |
| | CHO-S-fasMUC1 | 0.91 | No Binding |
| | CHO-S-hEGFR | 0.66 | No Binding |
| | CHO-S-fasEGFR | 0.19 | No Binding |
| ISO | CHO-S- | 4.05 | No Binding |
| | hMUC1(484AA) | | |
| | CHO-S-fasMUC1 | 1.15 | No Binding |
| | CHO-S-hEGFR | 0.70 | No Binding |
| | CHO-S-fasEGFR | 0.58 | No Binding |
| E-6C4-10M1 | CHO-S- | 92.6 | Binding |
| | hMUC1(484AA) | | |
| | CHO-S-fasMUC1 | 46.0 | Binding |
| | CHO-S-hEGFR | 51.0 | Binding |
| | CHO-S-fasEGFR | 18.0 | Binding |
| E-9A6-10M1 | CHO-S- | 91.4 | Binding |
| | hMUC1(484AA) | | |
| | CHO-S-fasMUC1 | 45.7 | Binding |
| | CHO-S-hEGFR | 61.9 | Binding |
| | CHO-S-fasEGFR | 22.3 | Binding |
| E-9D2-10M1 | CHO-S- | 93.0 | Binding |
| | hMUC1(484AA) | | |
| | CHO-S-fasMUC1 | 44.8 | Binding |
| | CHO-S-hEGFR | 50.7 | Binding |
| | CHO-S-fasEGFR | 18.7 | Binding |
| E-9F3-10M1 | CHO-S- | 94.0 | Binding |
| | hMUC1(484AA) | | |
| | CHO-S-fasMUC1 | 45.2 | Binding |
| | CHO-S-hEGFR | 60.4 | Binding |
| | CHO-S-fasEGFR | 22.1 | Binding |
| E-9H2-10M1 | CHO-S- | 95.0 | Binding |
| | hMUC1(484AA) | | |
| | CHO-S-fasMUC1 | 60.3 | Binding |
| | CHO-S-hEGFR | 50.7 | Binding |
| | CHO-S-fasEGFR | 18.8 | Binding |
| E-6C4-M-11E8 | CHO-S- | 94.0 | Binding |
| | hMUC1(484AA) | | |
| | CHO-S-fasMUC1 | 48.2 | Binding |
| | CHO-S-hEGFR | 48.1 | Binding |
| | CHO-S-fasEGFR | 18.3 | Binding |
| E-9A6-M-11E8 | CHO-S- | 93.5 | Binding |
| | hMUC1(484AA) | | |
| | CHO-S-fasMUC1 | 45.1 | Binding |
| | CHO-S-hEGFR | 63.3 | Binding |
| | CHO-S-fasEGFR | 23.0 | Binding |
| E-9D2-M-11E8 | CHO-S- | 93.8 | Binding |
| | hMUC1(484AA) | | |
| | CHO-S-fasMUC1 | 47.0 | Binding |
| | CHO-S-hEGFR | 50.1 | Binding |
| | CHO-S-fasEGFR | 16.8 | Binding |
| E-9F3-M-11E8 | CHO-S- | 94.2 | Binding |
| | hMUC1(484AA) | | |
| | CHO-S-fasMUC1 | 45.5 | Binding |
| | CHO-S-hEGFR | 58.8 | Binding |
| | CHO-S-fasEGFR | 20.5 | Binding |
| E-9H2-M-11E8 | CHO-S- | 94.0 | Binding |
| | hMUC1(484AA) | | |
| | CHO-S-fasMUC1 | 47.0 | Binding |
| | CHO-S-hEGFR | 49.3 | Binding |
| | CHO-S-fasEGFR | 15.1 | Binding |

### Binding Affinity of Anti-EGFR/MUC1 Bispecific Antibodies

The binding affinity of anti-EGFR/MUC1 bispecific antibodies to His-tagged human EGFR proteins (hEGFR-His, ACROBiosystems Inc., Cat #: EGR-H5222), His-tagged monkey EGFR proteins (fasEGFR-His, ACROBiosystems Inc., Cat #: EGR-C52H1), His-tagged human MUC1 proteins (hMUC1(24-1158)-His, including positions 24-1158 of human MUC1 (SEQ ID NO: 59)), His-tagged human MUC1 proteins (hMUC1(961-1152)-His, including positions 961-1152 of human MUC1(SEQ ID NO: 59)), and His-tagged monkey MUC1 (fasMUC1-His, SEQ ID NO: 60) were verified by surface plasmon resonance (SPR) using Biacore™ (Biacore, Inc., Piscataway N.J.) 8K biosensor equipped with pre-immobilized Protein A sensor chips.

Specifically, his-tagged proteins were diluted to 200 nM, with 1×HBS-EP+buffer (PH 7.4) and then injected into the Biacore™ 8K biosensor at 10 μL/min for about 50 seconds to achieve a desired protein density (e.g., about 100 response units (RU)). Purified antibodies at 1 μg/mL in 1× HBS-EP+buffer (PH 7.4) were then injected at 10 μL/min for 50 seconds.

Dissociation was monitored for 400 seconds. The chip was regenerated after the last injection of each titration with a glycine solution (pH 1.5) at 30 L/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

Table 2 Antibody Protein kon (1/Ms) koff (1/s) KD (M) fasEGFR-His 4.74E+05 1.56E-02 3.29E-08 hEGFR-His 8.34E+06 2.39E-01 2.86E-08 E-6C4-10M1 hMUC1(24-1158)-His 4.45E+04 6.61E-04 1.49E-08 hMUC(961-1152)-1.72E+05 8.01E-04 4.65E-09 His fasMUC1-His 1.73E+05 4.69E-04 2.72E-09 E-9A6-10M1 fasEGFR-His 2.03E+05 1.15E-03 5.63E-09 hEGFR-His 1.79E+05 1.01E-03 5.65E-09

TABLE 2

| Antibody | Protein | kon (1/Ms) | koff (1/s) | KD (M) |
|---|---|---|---|---|
| E-6C4-10M1 | fasEGFR-His | 4.74E+05 | 1.56E−02 | 3.29E−08 |
| | hEGFR-His | 8.34E+06 | 2.39E−01 | 2.86E−08 |
| | hMUC1(24-1158)-His | 4.45E+04 | 6.61E−04 | 1.49E−08 |
| | hMUC1(961-1152)- | 1.72E+05 | 8.01E−04 | 4.65E−09 |
| | His | | | |
| | fasMUC1-His | 1.73E+05 | 4.69E−04 | 2.72E−09 |
| E-9A6-10M1 | fasEGFR-His | 2.03E+05 | 1.15E−03 | 5.63E−09 |
| | hEGFR-His | 1.79E+05 | 1.01E−03 | 5.65E−09 |
| | hMUC1(24-1158)-His | 4.41E+04 | 1.19E−03 | 2.69E−08 |
| | hMUC1(961-1152)- | 1.37E+05 | 1.77E−03 | 1.29E−08 |
| | His | | | |
| | fasMUC1-His | 2.58E+05 | 1.29E−03 | 4.99E−09 |
| E-9D2-10M1 | fasEGFR-His | 9.30E+05 | 4.13E−02 | 4.44E−08 |
| | hEGFR-His | 3.02E+05 | 1.81E−02 | 6.00E−08 |
| | hMUC1(24-1158)-His | 6.20E+04 | 1.48E−03 | 2.38E−08 |
| | hMUC1(961-1152)- | 2.78E+05 | 2.21E−03 | 7.96E−09 |
| | His | | | |
| | fasMUC1-His | 2.66E+05 | 1.35E−03 | 5.09E−09 |
| E-9F3-10M1 | fasEGFR-His | 5.46E+04 | 1.69E−03 | 3.10E−08 |
| | hEGFR-His | 5.99E+04 | 1.86E−03 | 3.11E−08 |
| | hMUC1(24-1158)-His | 5.34E+04 | 1.28E−03 | 2.40E−08 |
| | hMUC1(961-1152)- | 2.36E+05 | 1.73E−03 | 7.31E−09 |
| | His | | | |
| | fasMUC1-His | 2.25E+05 | 1.04E−03 | 4.61E−09 |
| E-9H2-10M1 | fasEGFR-His | 7.20E+05 | 3.39E−02 | 4.70E−08 |
| | hEGFR-His | 2.28E+05 | 1.67E−02 | 7.32E−08 |
| | hMUC1(24-1158)-His | 4.95E+04 | 7.00E−04 | 1.41E−08 |
| | hMUC1(961-1152)- | 1.80E+05 | 8.15E−04 | 4.54E−09 |
| | His | | | |
| | fasMUC1-His | 1.80E+05 | 4.89E−04 | 2.72E−09 |
| E-6C4-M-11E8 | fasEGFR-His | 3.67E+05 | 2.91E−02 | 7.93E−08 |
| | hEGFR-His | 2.87E+05 | 2.64E−02 | 9.18E−08 |
| | hMUC1(24-1158)-His | 9.27E+04 | 5.75E−04 | 6.21E−09 |
| | hMUC1(961-1152)- | 4.10E+05 | 1.09E−03 | 2.66E−09 |
| | His | | | |
| | fasMUC1-His | 3.98E+05 | 1.02E−02 | 2.57E−08 |
| E-9A6-M-11E8 | fasEGFR-His | 2.13E+05 | 1.16E−03 | 5.44E−09 |
| | hEGFR-His | 1.83E+05 | 1.01E−03 | 5.54E−09 |
| | hMUC1(24-1158)-His | 7.91E+04 | 1.37E−03 | 1.73E−08 |
| | hMUC1(961-1152)- | 2.18E+05 | 2.18E−03 | 9.99E−09 |
| | His | | | |
| | fasMUC1-His | 4.01E+05 | 1.62E−02 | 4.05E−08 |
| E-9D2-M-11E8 | fasEGFR-His | 4.76E+05 | 2.48E−02 | 5.22E−08 |
| | hEGFR-His | 3.37E+05 | 2.00E−02 | 5.95E−08 |
| | hMUC1(24-1158)-His | 9.27E+04 | 5.54E−04 | 5.97E−09 |
| | hMUC1(961-1152)- | 1.95E+05 | 5.43E−04 | 2.79E−09 |
| | His | | | |
| | fasMUC1-His | 4.70E+05 | 8.96E−03 | 1.91E−08 |
| E-9F3-M-11E8 | fasEGFR-His | 6.71E+04 | 1.93E−03 | 2.87E−08 |
| | hEGFR-His | 7.07E+04 | 2.03E−03 | 2.87E−08 |
| | hMUC1(24-1158)-His | 1.04E+05 | 5.43E−04 | 5.24E−09 |
| | hMUC1(961-1152)- | 3.36E+05 | 8.81E−04 | 2.63E−09 |
| | His | | | |
| | fasMUC1-His | 4.26E+05 | 1.10E−02 | 2.57E−08 |
| E-9H2-M-11E8 | fasEGFR-His | 2.15E+05 | 1.73E−02 | 8.01E−08 |
| | hEGFR-His | 1.78E+05 | 1.57E−02 | 8.83E−08 |
| | hMUC1(24-1158)-His | 4.00E+05 | 1.01E−02 | 2.54E−08 |
| | hMUC1(961-1152)- | 9.23E+04 | 5.71E−04 | 6.19E−09 |
| | His | | | |
| | fasMUC1-His | 3.89E+05 | 1.05E−03 | 2.69E−09 |

The results showed that the anti-EGFR/MUC1 bispecific antibodies E-6C4-COM, E-9A6-10M1, E-9D2-10M1, E-9F3-10M1, E-91H2-10M1, E-6C4-M-11E8, E-9A6-M-11E8, E-9D2-M-11E8, E-9F3-M-11E8 and E-91H2-M-11E8 can all bind to human EGFR, human MUC1, monkey EGFR and monkey MUC1.

Internalization of Antibodies Targeting EGFR and/or MUC1

Anti-EGFR antibodies, anti-MUC antibodies, anti-EGFR/MUC1 bispecific antibodies, anti-MGFR/CD28 bispecific antibodies or anti-MUC/CD28 bispecific antibodies (2.5 g/mL) together with the pHAb-Goat anti-human IgG secondary antibody were added to NUCG-4 cells (Cobioer, Reference No.:CBP60493), Pane 02.03 cells (ATCC, Reference No.: CRL-2553) or HCC7 cells (ATCC, Reference No.: CRL-23 15), respectively, and incubated for 6 hour. The cells were centrifuged and washed with FACS buffer. MFI was measured using a flow cytometer. Endocytosis rates of the antibodies were calculated. For isotype control (ISO), an antibody targeting an irrelevant target protein was used. The results are shown in the following table.

TABLE 3

| | NUCG-4 | | Panc 02.03 | | HCC70 | |
|---|---|---|---|---|---|---|
| | | Positive | | Positive | | Positive |
| | | Popu- | | Popu- | | Popu- |
| | | lation | | lation | | lation |
| Antibodies | MFI | (%) | MFI | (%) | MFI | (%) |
| ISO | 6181 | 1.00 | 17060 | 8.80 | 12993 | 3.41 |
| NC ± Secondary | 5849 | 0.24 | 8875 | 1.25 | 11966 | 2.30 |
| antibody | | | | | | |
| Cetuximab | 21462 | 85.9 | 60940 | 95.5 | 48182 | 93.6 |
| analog | | | | | | |
| 1H7 analog | 19714 | 74.5 | 16072 | 14.7 | 29926 | 62.5 |
| Gatipotuzumab | 16032 | 37.7 | 27362 | 33.1 | 127449 | 74.9 |
| analog | | | | | | |
| E−6C4 | 23876 | 75.2 | 52057 | 92.0 | 45555 | 90.2 |
| 10M1 | 21686 | 79.7 | 20026 | 32.6 | 38181 | 81.7 |
| M-11E8 | 23439 | 83.8 | 22906 | 35.8 | 28052 | 51.6 |
| CD28-E−6C4 | 18233 | 53.6 | 39186 | 78.1 | 37617 | 73.0 |
| 10M1-CD28 | 7643 | 2.01 | 10413 | 1.86 | 16226 | 5.01 |
| M-11E8-CD28 | 8138 | 4.22 | 10959 | 1.96 | 15034 | 3.78 |
| E−6C4-10M1 | 32597 | 96.9 | 54349 | 93.8 | 64446 | 97.0 |
| E−9A6-10M1 | 31885 | 97.4 | 66227 | 96.9 | 60655 | 96.9 |
| E−9D2-10M1 | 39853 | 98.7 | 53681 | 93.1 | 64400 | 97.1 |
| E−9F3-10M1 | 35228 | 98.1 | 59896 | 95.6 | 67730 | 97.8 |
| E−9H2-10M1 | 30659 | 95.6 | 50169 | 92.1 | 57950 | 96.9 |
| E−6C4-M-11E8 | 38517 | 97.4 | 57679 | 91.8 | 50308 | 93.1 |
| E−9A6-M-11E8 | 38776 | 98.3 | 77263 | 94.6 | 59699 | 95.2 |
| E−9D2-M-11E8 | 42144 | 98.8 | 71531 | 95.2 | 49014 | 92.4 |
| E−9F3-M-11E8 | 31639 | 96.8 | 63632 | 94.3 | 43069 | 86.6 |
| E−9H2-M-11E8 | 32499 | 95.8 | 58288 | 94.7 | 42902 | 88.9 |

Cetuximab is an EGFR-targeting chimeric monoclonal IgG1 antibody originally developed by ImClone Systems and first launched in Switzerland in 2003 as Erbitux™ by Merck KGaA as a monotherapy and in combination with irinotecan for the treatment of irinotecan-refractory metastatic colorectal cancer. The heavy chain and light chain sequences of Cetuximab analog are shown in SEQ ID NO: 61 and SEQ ID NO: 62, respectively. 1H7 is a murine monoclonal IgG antibody targeting the extracellular region of human MUC1 C-terminal subunit, developed by Chungbuk National University and Peptron Co. Ltd.

The heavy chain variable region and light chain variable region of 1H7 are shown in SEQ ID NO: 63 and SEQ ID NO: 64 respectively.

Gatipotuzumab, a glyco-engineered humanized monoclonal antibody that recognizes the tumor-associated epitope of MUC1, is in phase II clinical development at Glycotope for the treatment of recurrent epithelial ovarian, fallopian tube or primary peritoneal cancers. The heavy chain variable region and light chain variable region of Gatipotuzumab are shown in SEQ ID NO: 65 and SEQ ID NO: 66 respectively.

The data showed that E-6C4-10M1, E-9A6-10M1, E-9D2-10M1, E-9F3-10M1, E-9H2-1OM1, E-6C4-M-11E8, E-9A6-M-11E8, E-9D2-M-11E8, E-9F3-M-11E8 and E-9H2-M-11E8 had higher cell endocytosis rates compared with positive control Cetuximab analog, 1H7 analog and Gatipotuzumab analog in NUCG-4 cells. E-6C4-10M1 and E-6C4-M-11E8 had higher cell endocytosis rates compared with corresponding monoclonal antibody E-6C4, 1OM1 and M-11E8 and corresponding monovalent antibody CD28-E-6C4, 1OM1-CD28 and M-11E8-CD28.

Binding Activity Verification of Antibodies Targeting EGFR and/or MUC1

The binding activities of anti-EGFR antibodies, anti-MUC1 antibodies, anti-EGFR/MUC1 bispecific antibodies, anti-EGFR/CD28 bispecific antibodies or anti-MUC1/CD28 bispecific antibodies to HCC827 cells (ATCC, Reference No.: CRL-2868), HCC70 cells, A431 cells (ATCC, Reference No.: CRL-1555) and ZR-75-1 cells (ATCC, Reference No.: CRL-1500) cells were verified by flow cytometry. The secondary antibody used in the experiment was: Alexa Fluor®647 anti-human IgG Fey (Jackson ImmunoResearch Laboratories, Inc., Cat #:109-606-170). EC50 was determined using serially diluted sample antibodies (maximum concentration: 20 μg/mL, 2-fold dilutions, 11 gradients). The results are shown in the table below.

TABLE 4

| | EC50 (μg/mL) | | | |
|---|---|---|---|---|
| | HCC827 | HCC70 | A431 | ZR-75-1 |
| Antibody | cells | cells | cells | cells |
| 1H7 analog | 2.97 | 10.83 | 2.31 | 3.88 |
| Gatipotuzumab analog | 29.06 | 0.23 | 63.80 | 11.38 |
| E-6C4-10M1 | 0.48 | 0.39 | 3.30 | 1.81 |
| E-9A6-10M1 | 1.23 | 1.52 | 1.48 | 4.28 |
| E-9D2-10M1 | 0.53 | 0.37 | 2.47 | 1.50 |
| E-9F3-10M1 | 0.74 | 0.84 | 0.89 | 2.50 |

### Example 2. Antibody Drug Conjugates (ADC)

After Protein A purification, antibodies E-6C4-10M1, E-9A6-10M1, E-9D2-10M1, E-9F3-10M1, E-6C4, E-9A6, E-9D2, E-9F3 and 10M1 were dialyzed and concentrated in PBS buffer by ultrafiltration. The concentration was determined by UV absorption. These antibodies were used for the subsequent antibody drug coupling reactions.

Coupling of Antibodies with Drug Molecules

Each purified antibodies was coupled with MMAE (monomethyl auristatin E) or MMAF (monomethyl auristatin F) through a maleimidocaproyl-valine-citrulline-p-aminobenzyloxycarbonyl (VC) linker.

For the names of antibody-drug conjugates, “ADC” is added directly after the antibody name. For example, if E-6C4-10M1 is coupled to MMAE, it is named as E-6C4-10M1-ADC.

HIC-HPLC were used to detect the coupling of antibodies with drug molecules. The HIC-HPLC detection results showed that the drug-to-antibody ratio (DAR) of ADC is about 4. For isotype control, a human IgG1 isotype control was coupled to MMAE to form isotype-ADC (ISO-ADC).

### In Vitro Killing Activity

Different concentrations of ADCs (10 μg/mL, 3.33 μg/mL, 1.11 μg/mL, 0.37 μg/mL, 0.123 μg/mL, 0.041 μg/mL, 0.013 μg/mL, and 0.004 μg/mL) were used to treat human lung cancer cell line NCI-H1975 (5×10³) or pancreatic carcinoma cell line Panc.02.03 (5×10³) cultured in a cell culture plate, and the killing activity was detected after 72 hours of incubation in IncuCyte (Sartorius AG, IncuCyte® S3). The results are shown in the table below.

TABLE 5

IC50 (μg/mL)

| Antibodies | NCI-H1975 | Panc.02.03 |
|---|---|---|
| E-6C4-ADC | 0.0520 | 0.1132 |
| 10M1-ADC | Not detected | 4.4850 |
| E-6C4-10M1-ADC | 0.6336 | 0.6265 |
| CD28-E-6C4-ADC | 0.6336 | 0.9297 |
| Cetuximab analog | 0.0540 | 0.2282 |

The results showed that the cytotoxic potency of monovalent ADC (CD28-E-6C4-ADC) was reduced in comparison with monoclonal anti-EGFR ADC (E-6C4-ADC) (the killing activity of CD28-E-6C4-ADC is 8~12 fold lower than that of anti-EGFR ADC in Panc.02.03 cells and NCI-H1975 cells), suggesting that E-6C4-10M1-ADC has a potential to increase tumor cell specificity to minimize skin toxicity due to ubiquitous basal EGFR expression.

### Example 3. Anti-Tumor Activity in NUGC-4 Xenograft Model

The ADCs were tested for their effects on tumor growth in vivo in a model of gastric cancer. About 5×10⁶ NUGC-4 cells were injected subcutaneously in B-NDG mice (Biocytogen Pharmaceuticals (Beijing) Co., Ltd., Cat #: B-CM-002). When the tumor size reached about 200 mm³, the mice were randomly placed into treatment or control groups based on tumor size. The mice were then injected with phosphate buffer saline (PBS) or ADCs by intravenous (i.v.) administration. The frequency of administration was once a week (l administrations in total). Details are shown in the table below.

TABLE 6

| | | | | | | Total No. of |
|---|---|---|---|---|---|---|
| Group | No. of mice | ADC | Dosage | Route | Frequency | administration |
| G1 | 5 | PBS | + 13 | i.v. | QW | 1 |
| G2 | 5 | Cetuximab analog-ADC | 3 mg/kg | i.v. | QW | 1 |
| G3 | 5 | 1H7 analog-ADC | 3 mg/kg | i.v. | QW | 1 |
| G4 | 5 | Gatipotuzumab analog- | 3 mg/kg | i.v. | QW | 1 |
| | | ADC | | | | |
| G5 | 5 | E−9A6-10M1-ADC | 3 mg/kg | i.v. | QW | 1 |
| G6 | 5 | E−6C4-10M1-ADC | 3 mg/kg | i.v. | QW | 1 |
| G7 | 5 | E−9A6-ADC | 3 mg/kg | i.v. | QW | 1 |
| G8 | 5 | E−6C4-ADC | 3 mg/kg | i.v. | QW | 1 |
| G9 | 5 | 10M1-ADC | 3 mg/kg | i.v. | QW | 1 |

The lengths of the long axis and the short axis of the tumor were measured and the volume of the tumor was calculated as 0.5×(long axis)×(short axis)². The tumor growth inhibition (TGI) was calculated using the following formula: TGI (%)=[r-(Ti-TO)/(Vi-V0)]×100. Ti is the average tumor volume in the treatment group on day i. TO is the average tumor volume in the treatment group on day zero. Vi is the average tumor volume in the control group on day i. V0 is the average tumor volume in the control group on day zero. T-test was perfo1med for statistical analysis. A TGI higher than 60% indicates clear suppression of tumor growth. P<0.05 is a threshold to indicate a significant difference.

During the experimental period, little difference was observed in the body weights of mice in different groups. The table below summarizes the results for this experiment, including the tumor volumes on the day of grouping (Day 0), 7 days after the grouping (Day 7), and at the end of the experiment (Day 14); the survival rate of the mice; TGI (%) and the statistical differences (P value) of tumor volumes between the treatment and control groups.

TABLE 7

| | Tumor volume (mm³) | | | | |
|---|---|---|---|---|---|
| Group | Day 0 | Day 7 | Day 14 | TGI (%) | P value |
| G1 | 185 ± 5 | 303 ± 42 | 510 ± 65 | NA | NA |
| G2 | 185 ± 16 | 240 ± 21 | 408 ± 55 | 31.2 | 0.267 |
| G3 | 185 ± 14 | 188 ± 30 | 297 ± 57 | 65.6 | 0.039 |
| G4 | 185 ± 13 | 175 ± 28 | 286 ± 35 | 68.9 | 0.016 |
| G5 | 185 ± 7 | 178 ± 19 | 256 ± 15 | 78.3 | 0.005 |
| G6 | 185 ± 8 | 148 ± 16 | 219 ± 14 | 89.4 | 0.002 |
| G7 | 185 ± 16 | 161 ± 18 | 330 ± 52 | 55.4 | 0.064 |
| G8 | 185 ± 11 | 178 ± 23 | 309 ± 48 | 62.0 | 0.038 |
| G9 | 185 ± 8 | 166 ± 18 | 278 ± 38 | 71.3 | 0.015 |

The treatment groups showed different tumor inhibitory effects. Overall, the anti-tumor-activities of the bispecific antibody ADCs (GS-G6) are stronger than those of corresponding monoclonal antibody ADCs (G7-G9), and also stronger than those of positive controls (G2-G4).

In another similar experiment, the injected volume was calculated based on the weight of the mouse to reach a dose of 1.5 mg/kg, 3 mg/kg, or 5 mg/kg. The frequency of administration was once a week (2 administrations in total). Details and results are shown in the tables below.

TABLE 8

| | | | | | | Total No. of |
|---|---|---|---|---|---|---|
| Group | No. of mice | ADC | Dosage | Route | Frequency | administration |
| G1 | 5 | PBS | + 13 | i.v. | QW | 2 |
| G2 | 5 | ISO-ADC | 1.5 mg/kg | i.v. | QW | 2 |
| G3 | 5 | ISO-ADC | 3 mg/kg | i.v. | QW | 2 |
| G4 | 5 | ISO-ADC | 5 mg/kg | i.v. | QW | 2 |
| G5 | 5 | Cetuximab analog-ADC | 1.5 mg/kg | i.v. | QW | 2 |
| G6 | 5 | Cetuximab analog-ADC | 3 mg/kg | i.v. | QW | 2 |
| G7 | 5 | Cetuximab analog-ADC | 5 mg/kg | i.v. | QW | 2 |
| G8 | 5 | E−9A6-10M1-ADC | 1.5 mg/kg | i.v. | QW | 2 |
| G9 | 5 | E−9A6-10M1-ADC | 3 mg/kg | i.v. | QW | 2 |
| G10 | 5 | E−9A6-10M1-ADC | 5 mg/kg | i.v. | QW | 2 |
| G11 | 5 | E−9F3-10M1-ADC | 1.5 mg/kg | i.v. | QW | 2 |
| G12 | 5 | E−9F3-10M1-ADC | 3 mg/kg | i.v. | QW | 2 |
| G13 | 5 | E−9F3-10M1-ADC | 5 mg/kg | i.v. | QW | 2 |
| G14 | 5 | E−9D2-10M1-ADC | 1.5 mg/kg | i.v. | QW | 2 |
| G15 | 5 | E−9D2-10M1-ADC | 3 mg/kg | i.v. | QW | 2 |
| G16 | 5 | E−9D2-10M1-ADC | 5 mg/kg | i.v. | QW | 2 |
| G17 | 5 | E−6C4-10M1-ADC | 1.5 mg/kg | i.v. | QW | 2 |
| G18 | 5 | E−6C4-10M1-ADC | 3 mg/kg | i.v. | QW | 2 |
| G19 | 5 | E−6C4-10M1-ADC | 5 mg/kg | i.v. | QW | 2 |

Table 9 below summarizes the results for this experiment, including the tumor volumes on the day of grouping (Day 0), 11 days after the grouping (Day 11), and at the end of the experiment (Day 21); the survival rate of the mice; TGI (%); and the statistical differences (P value) of tumor volumes between the treatment and control groups.

TABLE 9

| | Tumor volume (mm³) | | | | TGI | |
|---|---|---|---|---|---|---|
| Group | Day 0 | Day 11 | Day 21 | Survival | (%) | P value |
| G1 | 204 ± 7 | 454 ± 31 | 812 ± 34 | 5/5 | NA | NA |
| G2 | 204 ± 10 | 448 ± 55 | 839 ± 125 | 5/5 | −4.4 | 0.841 |
| G3 | 204 ± 13 | 400 ± 98 | 676 ± 187 | 5/5 | 22.3 | 0.496 |
| G4 | 204 ± 11 | 332 ± 68 | 540 ± 114 | 5/5 | 44.8 | 0.039 |
| G5 | 204 ± 11 | 538 ± 102 | 998 ± 142 | 5/5 | −30.7 | 0.237 |
| G6 | 204 ± 9 | 299 ± 24 | 685 ± 147 | 5/5 | 20.9 | 0.421 |
| G7 | 204 ± 10 | 162 ± 26 | 328 ± 69 | 5/5 | 79.7 | 2.343E−04 |
| G8 | 204 ± 12 | 361 ± 23 | 824 ± 64 | 5/5 | −2.1 | 0.869 |
| G9 | 204 ± 13 | 145 ± 15 | 195 ± 34 | 5/5 | 101.5 | 1.183E−06 |
| G10 | 204 ± 12 | 43 ± 4 | 31 ± 13 | 5/5 | 128.4 | 2.260E−08 |
| G11 | 204 ± 11 | 268 ± 17 | 545 ± 26 | 5/5 | 43.9 | 2.415E−04 |
| G12 | 204 ± 10 | 85 ± 8 | 159 ± 58 | 5/5 | 107.4 | 1.006E−05 |
| G13 | 204 ± 10 | 38 ± 3 | 12 ± 3 | 5/5 | 131.5 | 1.087E−08 |
| G14 | 204 ± 10 | 194 ± 31 | 406 ± 55 | 5/5 | 66.8 | 2.319E−04 |
| G15 | 204 ± 11 | 58 ± 13 | 26 ± 9 | 5/5 | 129.3 | 1.607E−08 |
| G16 | 204 ± 9 | 41 ± 12 | 12 ± 4 | 5/5 | 131.5 | 1.096E−08 |
| G17 | 204 ± 9 | 349 ± 101 | 525 ± 89 | 5/5 | 47.2 | 0.013 |
| G18 | 204 ± 10 | 64 ± 7 | 45 ± 12 | 5/5 | 126.1 | 2.378E−08 |
| G19 | 204 ± 9 | 42 ± 3 | 14 ± 4 | 5/5 | 131.2 | 1.129E−08 |

The treatment groups (G5-G19) showed better tumor inhibitory effects compared with the control groups (G1-G4), which were treated with PBS or ISO-ADC. Anti-EGFR/MUC1 bispecific antibody ADCs (G8-G19) showed better tumor inhibitory effects compared with the positive control groups (G5-G7) at the same dosage. Also, the treatment groups (G8-G19) showed dose-dependent anti-tumor activities.

### Example 4. Anti-Tumor Activity in HCC70 Xenograft Model

The ADCs were tested for their effect on tumor growth in vivo in a model of breast carcinoma. Specifically, about 1×10⁷ HCC70 cells were injected subcutaneously in each B-NDG mouse. When the tumors in the mice reached a volume of about 200 mm³, the mice were randomly placed into different groups based on the volume of the tumor (5 mice per group). The mice were then injected with phosphate buffer saline (PBS) or ADCs by intravenous (i.v.) administration. The frequency of administration was once a week (2 administrations in total). Details are shown in the table below.

TABLE 10

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | ISO-ADC | 3 mg/kg | i.v. | QW | 2 |
| G3 | 5 | Cetuximab analog-ADC | 3 mg/kg | i.v. | QW | 2 |
| G4 | 5 | 1H7 analog-ADC | 3 mg/kg | i.v. | QW | 2 |
| G5 | 5 | Gatipotuzumab analog- | 3 mg/kg | i.v. | QW | 2 |
| | | ADC | | | | |
| G6 | 5 | E−9A6-10M1-ADC | 3 mg/kg | i.v. | QW | 2 |
| G7 | 5 | E−9F3-10M1-ADC | 3 mg/kg | i.v. | QW | 2 |
| G8 | 5 | E−9D2-10M1-ADC | 3 mg/kg | i.v. | QW | 2 |
| G9 | 5 | E−6C4-10M1-ADC | 3 mg/kg | i.v. | QW | 2 |

The body weights of the mice were also measured twice a week. On the day of grouping (Day 0), the average body weight of each group was in the range of 22.6g-24.2g. At the end of the experiment (Day 25), the average weight of each group was in the range of 23.4g-25.2g.

Thus, the average weight change of each group was in the range of 100.9% o-10Q7%. The weights of mice in different groups all increased. The results showed that the tested ADCs were well tolerated and were not obviously toxic to the mice.

The table below summarizes the results for this experiment, including the tumor volumes on the day of grouping (Day 0), 14 days after grouping (Day 14) and at the end of the experiment (Day 25); the survival rate of the mice; TGI (%) and the statistical differences (P value) of tumor volumes and body weights between the treatment and control groups.

TABLE 11

| | | | | | P value | |
|---|---|---|---|---|---|---|
| | Tumor volume (mm³) | | | TGI | Body | Tumor |
| Group | Day 0 | Day 14 | Day 25 | (%) | weight | volume |
| G1 | 210 ± 8 | 478 ± 56 | 897 ± 76 | NA | NA | NA |
| G2 | 210 ± 12 | 448 ± 31 | 882 ± 39 | 2.3 | 0.487 | 0.860 |
| G3 | 210 ± 9 | 144 ± 22 | 114 ± 18 | 114.0 | 0.712 | 8.43E−06 |
| G4 | 210 ± 14 | 87 ± 17 | 116 ± 44 | 113.7 | 0.472 | 2.03E−05 |
| G5 | 210 ± 11 | 151 ± 44 | 122 ± 38 | 112.8 | 0.590 | 1.73E−05 |
| G6 | 210 ± 9 | 89 ± 13 | 46 ± 7 | 123.8 | 0.977 | 3.81E−06 |
| G7 | 210 ± 12 | 87 ± 15 | 26 ± 3 | 126.7 | 0.674 | 3.12E−06 |
| G8 | 210 ± 11 | 78 ± 10 | 32 ± 4 | 125.8 | 0.415 | 3.31E−06 |
| G9 | 210 ± 10 | 96 ± 17 | 31 ± 5 | 126.0 | 0.131 | 3.29E−06 |

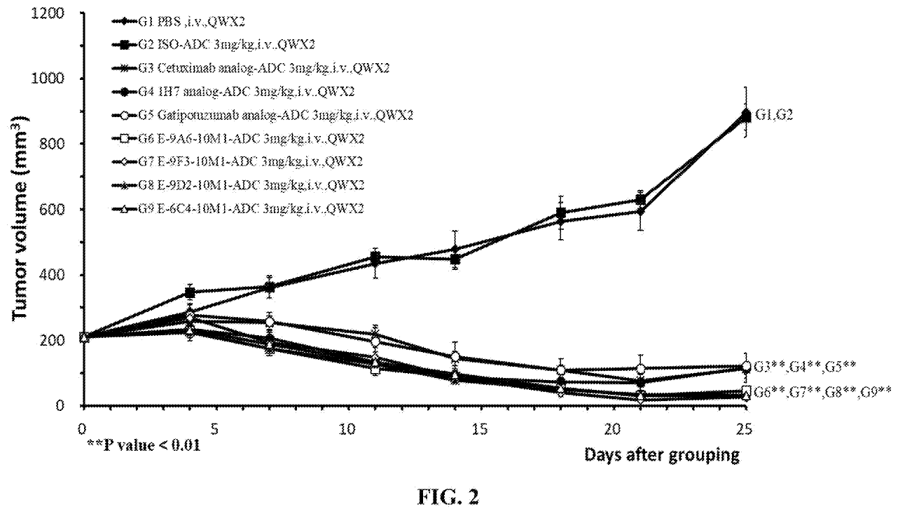

The tumor volumes of mice in different groups treated with the ADCs or PBS are shown in FIG. 2. The treatment groups (G3-G9) showed better tumor inhibitory effects compared with the control groups (G1-G2), which were treated with PBS or ISO-ADC. All four anti-EGFR/MUC1 bispecific antibody ADCs (G6-G9) at a dose of 3 mg/kg showed sustained and potent tumor suppression effects with high TGI. The TGI values of all tested EGFR/MUC1 bispecific antibody ADCs (G6-G9) were higher than those of the positive controls Cetuximab analog-ADC (G3), 1H7 analog-ADC (G4) and Gatipotuzumab analog-ADC (G5).

### Example 5. Anti-Tumor Activity in Panc 02.03 Xenograft Model

The ADCs were tested for their effect on tumor growth in vivo in a xenograft model of pancreatic adenocarcinoma. Specifically, about 2×10⁶ pancreatic adenocarcinoma epithelial Panc 02.03 cells were injected subcutaneously in each B-NDG mouse. When the tumors in the mice reached a volume of about 200 mm³, the mice were randomly placed into different groups based on tumor volume. The mice were then injected with PBS or ADCs by intravenous (i.v.) administration. The frequency of administration was once a week (2 administrations in total). Details are shown in the table below.

TABLE 12

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | ISO-ADC | 3 mg/kg | i.v. | QW | 2 |
| G3 | 5 | Cetuximab analog-ADC | 3 mg/kg | i.v. | QW | 2 |
| G4 | 5 | 1H7 analog-ADC | 3 mg/kg | i.v. | QW | 2 |
| G5 | 5 | Gatipotuzumab analog- | 3 mg/kg | i.v. | QW | 2 |
| | | ADC | | | | |
| G6 | 5 | E−9A6-10M1-ADC | 3 mg/kg | i.v. | QW | 2 |
| G7 | 5 | E−9F3-10M1-ADC | 3 mg/kg | i.v. | QW | 2 |
| G8 | 5 | E−9D2-10M1-ADC | 3 mg/kg | i.v. | QW | 2 |
| G9 | 5 | E−6C4-10M1-ADC | 3 mg/kg | i.v. | QW | 2 |

During the experimental period, little difference was observed in the body weights of mice in different groups. The table below summarizes the results for this experiment, including the tumor volumes on the day of grouping (Day 0), 17 days after grouping (Day 17) and at the end of the experiment (Day 27); the survival rate of the mice; TGI (%) and the statistical differences (P value) of body weights and tumor volumes between the treatment and control groups.

TABLE 13

| | | | | | | P value | |
|---|---|---|---|---|---|---|---|
| | Tumor volume (mm³) | | | | TGI | Body | Tumor |
| Group | Day 0 | Day 17 | Day 27 | Survival | (%) | weight | volume |
| G1 | 186 ± 6 | 725 ± 72 | 1435 ± 113 | 5/5 | NA | NA | NA |
| G2 | 186 ± 10 | 476 ± 44 | 1082 ± 128 | 5/5 | 28.3 | 0.433 | 0.073 |
| G3 | 186 ± 11 | 271 ± 14 | 591 ± 26 | 5/5 | 67.6 | 0.513 | 8.79E−05 |
| G4 | 185 ± 11 | 420 ± 25 | 896 ± 38 | 5/5 | 43.2 | 0.577 | 0.002 |
| G5 | 186 ± 13 | 437 ± 43 | 880 ± 56 | 5/5 | 44.5 | 0.975 | 0.002 |
| G6 | 186 ± 10 | 192 ± 23 | 418 ± 56 | 5/5 | 81.4 | 0.085 | 4.20E−05 |
| G7 | 186 ± 15 | 284 ± 45 | 558 ± 99 | 5/5 | 70.2 | 0.712 | 3.98E−04 |
| G8 | 186 ± 11 | 192 ± 36 | 350 ± 86 | 5/5 | 86.9 | 0.337 | 6.19E−05 |
| G9 | 186 ± 12 | 216 ± 17 | 527 ± 44 | 5/5 | 72.7 | 0.489 | 7.11E−05 |

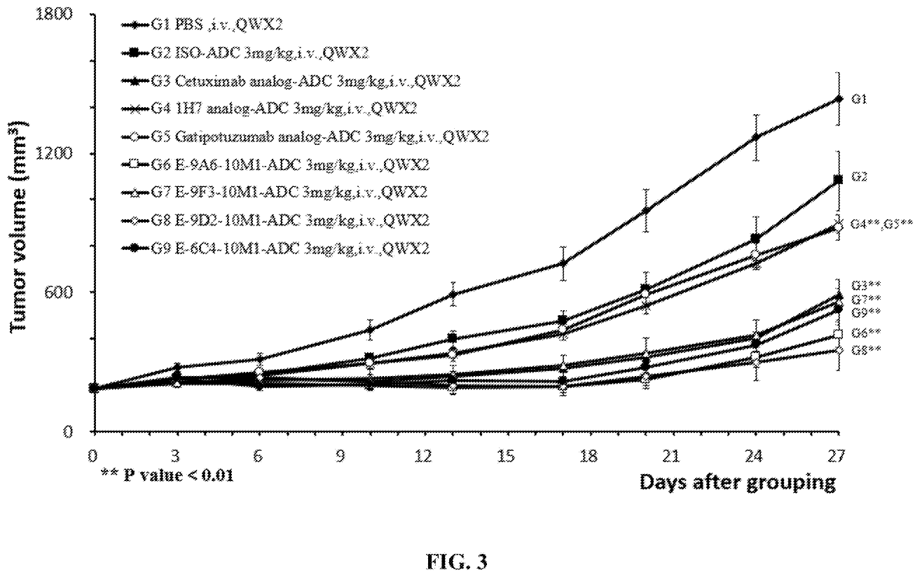

The tumor sizes in groups treated with the ADCs are shown in FIG. 3. The treatment groups (G3-G9) showed different tumor inhibitory effects. Overall, the ADCs (G6-G9) showed better anti-tumor activities compared with the positive control groups (G3-G5) at a dose of 3 mg/kg.

### Example 6. Anti-Tumor Activity in NCI-H1975 Xenograft Model

The ADCs were tested for their effects on tumor growth in vivo in a model of lung adenocarcinoma. Specifically, about 1×10⁶ NCI-H1975 cells were injected subcutaneously in each B-NDG mouse. When the tumors in the mice reached a volume of about 200 mm³, the mice were randomly placed into different groups based on the volume of the tumor (5 mice per group). The mice were then injected with PBS or ADCs by intravenous (i.v.) administration. The frequency of administration was once a week (2 administrations in total). Details are shown in the table below.

TABLE 14

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | Cetuximab analog-ADC | 3 mg/kg | i.v. | QW | 2 |
| G3 | 5 | 1H7 analog-ADC | 3 mg/kg | i.v. | QW | 2 |
| G4 | 5 | Gatipotuzumab analog-ADC | 3 mg/kg | i.v. | QW | 2 |
| G5 | 5 | E−9A6-10M1-ADC | 3 mg/kg | i.v. | QW | 2 |
| G6 | 5 | E−9F3-10M1-ADC | 3 mg/kg | i.v. | QW | 2 |
| G7 | 5 | E−9D2-10M1-ADC | 3 mg/kg | i.v. | QW | 2 |
| G8 | 5 | E−6C4-10M1-ADC | 3 mg/kg | i.v. | QW | 2 |

The table below summarizes the results for this experiment, including the tumor volumes on the day of grouping (Day 0), 10 days after grouping (Day 10) and at the end of the experiment (Day 21); TGI (%); and the statistical differences (P value) of tumor volumes between the treatment and control groups.

TABLE 15

| | Tumor volume (mm³) | | | | |
|---|---|---|---|---|---|
| Group | Day 0 | Day 10 | Day 21 | TGI (%) | P value |
| G1 | 192 ± 5 | 1071 ± 76 | 2490 ± 271 | NA | NA |
| G2 | 193 ± 11 | 232 ± 34 | 676 ± 236 | 78.9 | 0.001 |
| G3 | 193 ± 8 | 322 ± 28 | 854 ± 193 | 71.2 | 0.001 |
| G4 | 193 ± 9 | 412 ± 30 | 1267 ± 176 | 53.2 | 0.005 |
| G5 | 193 ± 8 | 181 ± 20 | 157 ± 24 | 101.5 | 1.3E−04 |
| G6 | 193 ± 13 | 188 ± 36 | 214 ± 107 | 99.1 | 5.3E−05 |
| G7 | 193 ± 6 | 180 ± 18 | 232 ± 127 | 98.3 | 6.7E−05 |
| G8 | 193 ± 5 | 213 ± 22 | 166 ± 37 | 101.2 | 2.9E−05 |

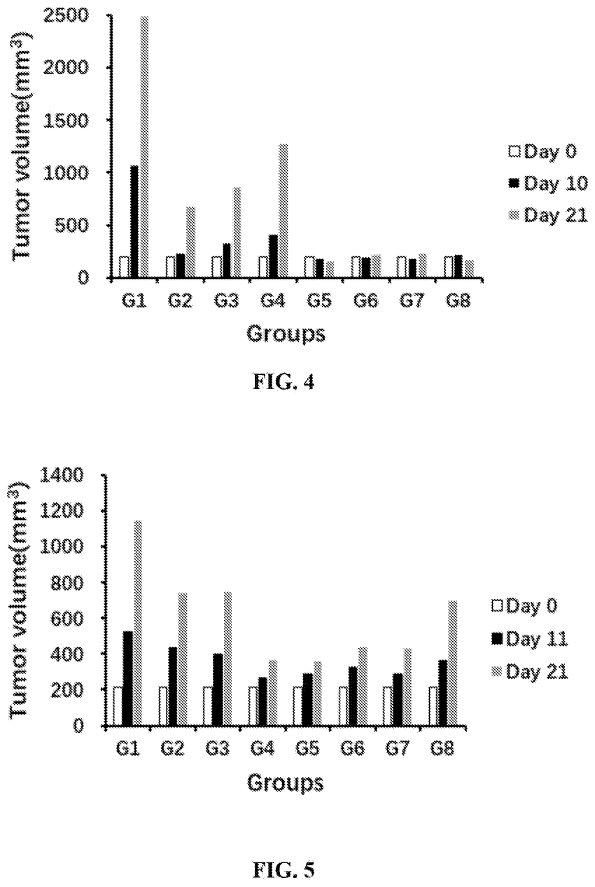

The tumor volumes of mice in different groups treated with the ADCs or PBS are shown in FIG. 4. The treatment groups (G2-G8) showed better tumor inhibitory effects compared with the control group (G1) treated with PBS. All four anti-EGFR/MUC1 bispecific antibody ADCs (G5-G8) at a dose of 3 mg/kg showed sustained and potent tumor suppression effects with a higher TGI than those of the positive controls (G2-G4).

### Example 7. Anti-Tumor Activity in NCI-H1650 Xenograft Model

The ADCs were tested for their effects on tumor growth in vivo in a model of lung cancer. About 1×10⁷ NCI-H1650 cells were injected subcutaneously in each B-NDG mouse. When the tumors in mice reached a volume of about 200 mm³, the mice were randomly placed into different groups based on the volume of the tumor. The mice were then injected with PBS, antibodies or ADCs by intravenous (i.v.) administration. The frequency of administration was once a week (2 administrations in total). Details are shown in the table below.

TABLE 16

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | 1H7 analog-ADC | 3 mg/kg | i.v. | QW | 2 |
| G3 | 5 | Gatipotuzumab analog- | 3 mg/kg | i.v. | QW | 2 |
| | | ADC | | | | |
| G4 | 5 | E−9A6-10M1-ADC | 3 mg/kg | i.v. | QW | 2 |
| G5 | 5 | E−6C4-10M1-ADC | 3 mg/kg | i.v. | QW | 2 |
| G6 | 5 | E−9A6-ADC | 3 mg/kg | i.v. | QW | 2 |
| G7 | 5 | E−6C4-ADC | 3 mg/kg | i.v. | QW | 2 |
| G8 | 5 | 10M1-ADC | 3 mg/kg | i.v. | QW | 2 |

During the experimental period, little difference was observed in the body weights of mice in different groups. The table below summarizes the results for this experiment, including the tumor volumes on the day of grouping (Day 0), 11 days after grouping (Day 11), and at the end of the experiment (Day 21); the survival rate of the mice; TGI (%); and the statistical differences (P value) of tumor volumes between the treatment and control groups.

TABLE 17

| | Tumor volume (mm³) | | | | TGI | |
|---|---|---|---|---|---|---|
| Group | Day 0 | Day 11 | Day 21 | Survival | (%) | P value |
| G1 | 212 ± 7 | 527 ± 34 | 1145 ± 86 | 5/5 | NA | NA |
| G2 | 213 ± 11 | 435 ± 28 | 741 ± 52 | 5/5 | 43.4 | 0.004 |
| G3 | 213 ± 10 | 397 ± 21 | 749 ± 36 | 5/5 | 42.5 | 0.003 |
| G4 | 213 ± 10 | 269 ± 13 | 362 ± 60 | 5/5 | 83.9 | 7.19E−05 |
| G5 | 213 ± 13 | 287 ± 22 | 360 ± 38 | 5/5 | 84.2 | 3.20E−05 |
| G6 | 213 ± 12 | 325 ± 9 | 443 ± 24 | 5/5 | 75.3 | 4.87E−05 |
| G7 | 213 ± 10 | 287 ± 33 | 429 ± 44 | 5/5 | 76.8 | 7.52E−05 |
| G8 | 213 ± 14 | 361 ± 42 | 702 ± 108 | 5/5 | 47.6 | 0.012 |

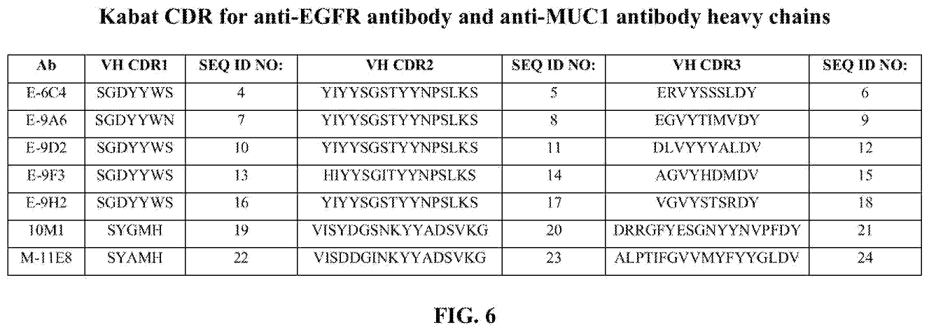

The tumor size in groups treated with the antibodies are shown in FIG. 5. The treatment groups showed different tumor inhibitory effects. Overall, the anti-tumor activities of the bispecific antibody ADCs (G4-G5) are stronger than those of corresponding monoclonal antibody ADCs (G6-G8), and stronger than those of the positive controls (G2-G3).

### Example 8. Anti-Tumor Activity in Patient Derived Lung Cancer Xenograft Model

The ADCs were tested for their effect on tumor growth in vivo in a xenograft model of lung cancer. Specifically, tumor fragments derived from a patient with lung cancer were inoculated subcutaneously in B-NDG mice. When the tumors in the mice reached a volume of about 250-300 mm³, the mice were randomly placed into different groups based on tumor volume. The mice were then injected with PBS or ADCs by intravenous (i.v.) administration. The frequency of administration was once a week (2 administrations in total). Details are shown in the table below.

TABLE 18

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | Cetuximab analog-ADC | 3 mg/kg | i.v. | QW | 2 |
| G3 | 5 | Gatipotuzumab analog- | 3 mg/kg | i.v. | QW | 2 |
| | | ADC | | | | |
| G4 | 5 | E−9F3-10M1-ADC | 3 mg/kg | i.v. | QW | 2 |
| G5 | 5 | E−6C4-10M1-ADC | 3 mg/kg | i.v. | QW | 2 |

The weights of mice in all treatment groups (G2-G5) all increased. The results showed that the tested antibodies were well tolerated and were not obviously toxic to the mice.

The table below summarizes the results for this experiment, including the tumor volumes on the day of grouping (Day 0), 10 days after grouping (Day 10) and at the end of the experiment (Day 21); the survival rate of the mice; TGI (%); and the statistical differences (P value) of tumor volumes between the treatment and control groups.

TABLE 19

| | Tumor volume (mm³) | | | | |
|---|---|---|---|---|---|
| Group | Day 0 | Day 10 | Day 21 | TGI (%) | P value |
| G1 | 256 ± 21 | 1191 ± 134 | 2206 ± 353 | NA | NA |
| G2 | 256 ± 28 | 324 ± 95 | 719 ± 172 | 76.3 | 0.005 |
| G3 | 257 ± 28 | 807 ± 181 | 1938 ± 468 | 13.8 | 0.676 |
| G4 | 256 ± 30 | 253 ± 76 | 325 ± 102 | 96.4 | 0.001 |
| G5 | 256 ± 27 | 292 ± 24 | 565 ± 215 | 84.1 | 0.004 |

The results showed that the anti-tumor activities of the bispecific antibody ADCs (G4-G5) at a dose of 3 mg/kg are stronger than those of the positive control groups (G2-G3).

### Example 9. In vivo efficacy in human ampullary carcinoma PDX model

The ADCs were tested for their effect in human ampullary carcinoma PDX model. Immunofluorescence staining of patient-derived ampullary tumor fragments was performed and the images were analyzed via HALO 3.2 version. The results showed that EGFR positive cell and MUC1 positive cell were 94.03% and 81.20% respectively.

B-NDG mice were engrafted in the right flank with patient-derived ampullary tumor fragments (2 mm×2 mm×2 mm). When the tumors in the mice reached a volume of about 250-300 mm³, the mice were randomly placed into different groups based on the volume of the tumor. The mice were then injected with PBS or ADCs by intravenous (i.v.) administration. Details of the administration scheme are shown in the table below.

TABLE 20

| | | | | | | Total No. of |
|---|---|---|---|---|---|---|
| Group | No. of mice | ADC | Dosage | Route | Frequency | administration |
| G1 | 5 | PBS | — | i.v. | QW | 2 |
| G2 | 5 | ISO-ADC | 3 mg/kg | i.v. | QW | 2 |
| G3 | 5 | 1H7 analog-ADC | 3 mg/kg | i.v. | QW | 2 |
| G4 | 5 | Gatipotuzumab analog-ADC | 3 mg/kg | i.v. | QW | 2 |
| G5 | 5 | Cetuximab analog-ADC | 3 mg/kg | i.v. | QW | 2 |
| G6 | 5 | E−6C4-10M1-ADC | 3 mg/kg | i.v. | QW | 2 |

The table below summarizes the results for this experiment, including the tumor volumes on the day of grouping (Day 0), 14 days after grouping (Day 14), 28 days after grouping (Day 28) and 42 days after grouping (Day 42); TGI (%); and the statistical differences (P value) of tumor volume between the treatment and control groups.

TABLE 21

| | Tumor volume (mm³) | | | | TGI | |
|---|---|---|---|---|---|---|
| Group | Day 0 | Day 14 | Day 28 | Day 42 | (%) | P value |
| G1 | 238 ± 14 | 691 ± 58 | 1231 ± 136 | 1867 ± 228 | NA | NA |
| G2 | 238 ± 21 | 549 ± 80 | 965 ± 186 | 1790 ± 370 | 4.7% | 0.864 |
| G3 | 238 ± 18 | 497 ± 114 | 973 ± 249 | 1885 ± 411 | −1.1% | 0.970 |
| G4 | 239 ± 26 | 608 ± 61 | 1284 ± 119 | 2349 ± 172 | −29.6% | 0.130 |
| G5 | 239 ± 17 | 348 ± 36 | 607 ± 110 | 1226 ± 152 | 39.4% | 0.047 |
| G6 | 239 ± 18 | 233 ± 54 | 234 ± 106 | 468 ± 198 | 85.9% | 0.002 |

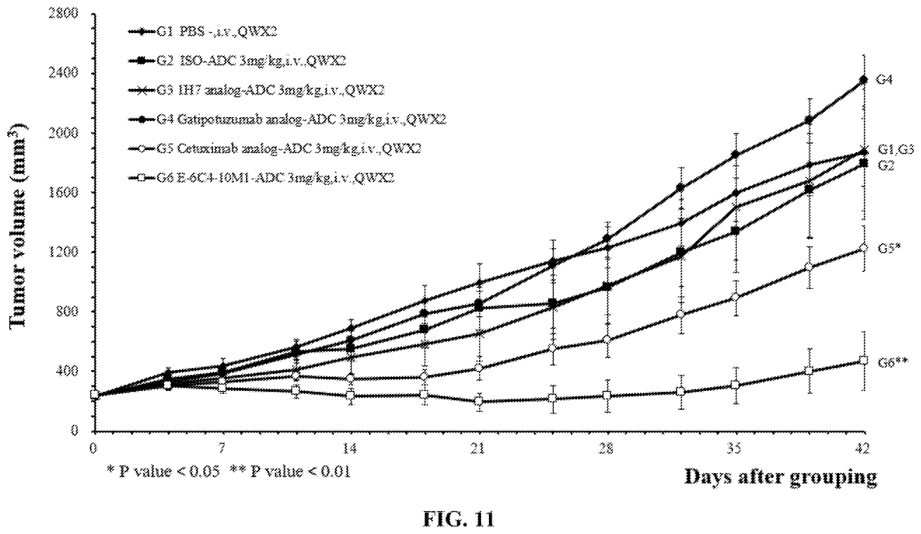

The tumor volume of mice in different groups are shown in FIG. 11. The results showed that the anti-tumor activities of E-6C4-10M1-ADC (G6) at a dose of 3 mg/kg are stronger than those of the positive control groups (G3-G5). In addition, the experiment was then continued until 60 days after grouping (Day 60) and the E-6C4-10M1-ADC (G6) still showed better tumor inhibitory effect than positive control groups (G3-G5) among which the mice of 1H7 analog-ADC and Gatipotuzumab analog-ADC treatment group (G3-G4) all died.

### Example 10. Internalization of Anti-EGFR/MUC1 Bispecific Antibody and ADC

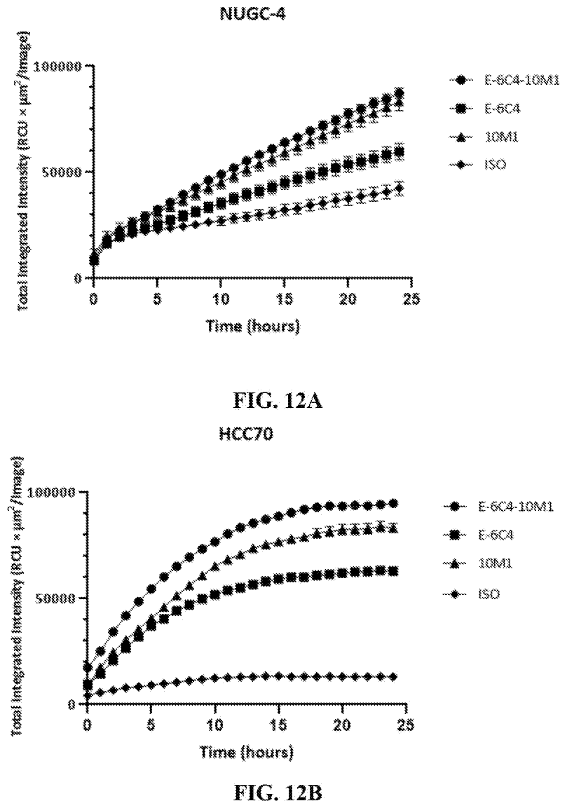

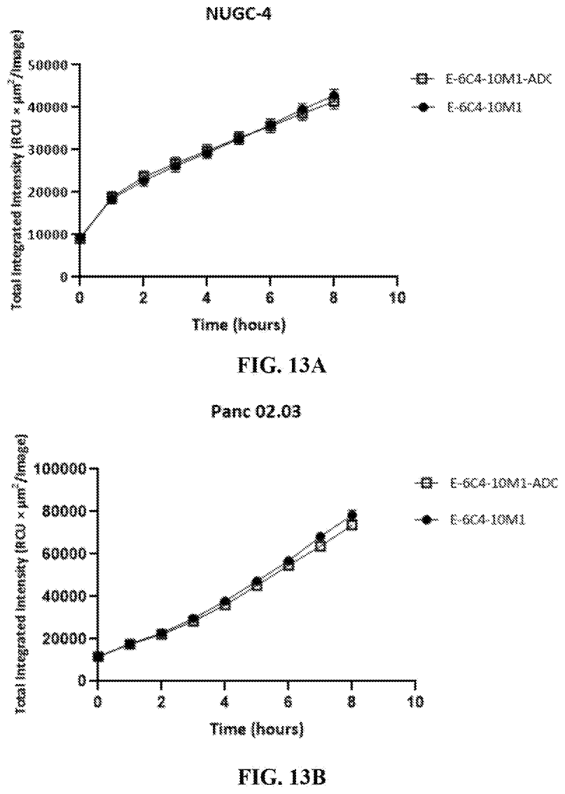

The anti-EGFR antibody, anti-MUC1 antibody, anti-EGFR/MUC1 bispecific antibody or anti-EGFR/MUC1 bispecific ADC was used to treat NUGC-4 cells, HCC70 cells or Pane 02.03 cells cultured in a cell culture plate, and the internalization activity was detected after 8 or 24 hours of incubation with IncuCyte (Sartorius AG, IncuCyte© S3). The results are shown in FIGS. 12A-12B and FIGS. 13A-13B.

As shown in FIGS. 12A-12B, E-6C4-10M1 exhibited better internalization effects than that of the corresponding parental monoclonal antibodies (E-6C4 and 10M1) in NUGC-4 cells and HCC70 cells. FIGS. 13A-13B indicated that the endocytosis activity of E-6C4-1OM I-ADC was not altered after the conjugation of MMAE.

### Example 11. Antibody Drug Conjugates

Coupling of Antibodies with Drug Molecules

The purified antibodies were coupled with CPT-1, CPT-2, CPT-3, or CPT-4 through a CPT-L linker. For the names of antibody-drug conjugates, CPTx (x=1, 2, 3, or 4) is added directly after the antibody name. For example, when E-6C4-10M1 is coupled to CPT-1, it is named as E-6C4-10M1-CPT1. As another example, when E-6C4-10M1 is coupled to CPT-2, it is named as E-6C4-10M1-CPT2. Exemplary ADCs obtained by this method included: E-6C4-CPT1, 1OM1-CPT1, E-6C4-10M1-CPT1, E-6C4-CPT2, 1OM1-CPT2, and E-6C4-10M1-CPT2.

For positive controls, 1H7 analog and Gatipotuzumab analog were coupled with CPT-2 through the CPT-L linker to obtain 1H7-CPT2 and Gatipotuzumab-CPT2 respectively. Gatipotuzumab analog was also coupled with GGFG-Dxd (Deruxtecan, MedChemExpress, Cat #: HY-13631E) to form Gatipotuzumab-Dxd.

MS (Mass Spectrometry) was used to detect the coupling of antibodies with drug molecules. The MS detection results showed that the DAR of the ADCs was about 8.

### Anti-Tumor Activity in Patient-Derived Pancreatic Cancer Xenograft Model

B-NDG mice were engrafted in the right flank with pancreatic cancer patient-derived tumor tissue fragments (2 mm×2 mm×2 mm). The immunofluorescence staining results showed that EGFR-positive cells and MUC1-positive cells in the pancreatic tumor fragments were 60.62% and 64.77%, respectively. When the tumors in the mice reached a volume of about 200-300 mm³, the mice were randomly placed into different groups based on the tumor volume. The mice were then injected with PBS or ADCs by i.v. administration. Details are shown in the table below.

TABLE 22

| | | | | | | Total No. of |
|---|---|---|---|---|---|---|
| Group | No. of mice | ADC | Dosage | Route | Frequency | administration |
| G1 | 5 | PBS | — | i. v. | QW | 2 |
| G2 | 5 | E−6C4-10M1-CPT2 | 3 mg/kg | i.v. | QW | 2 |
| G3 | 5 | E−6C4-10M1-CPT2 | 6 mg/kg | i.v. | QW | 2 |
| G4 | 5 | E−6C4-CPT2 | 3 mg/kg | i.v. | QW | 2 |
| | | 10M1-CPT2 | 3 mg/kg | | | |
| G5 | 5 | 1H7-CPT2 | 6 mg/kg | i.v. | QW | 2 |
| G6 | 5 | Gatipotuzumab-CPT2 | 6 mg/kg | i.v. | QW | 2 |
| G7 | 5 | Gatipotuzumab-Dxd | 6 mg/kg | i.v. | QW | 2 |

The table below summarizes the results for this experiment, including the tumor volumes on the day of grouping (Day 0), 14 days after grouping (Day 14) and 39 days after grouping (Day 39); TGT (%) and the statistical differences (P value) of tumor volume between the treatment and control groups.

TABLE 23

| | Tumor volume (mm³) | | | TGI (%) | P value |
|---|---|---|---|---|---|
| Group | Day 0 | Day 14 | Day 39 | (Day 39) | (Day 39) |
| G1 | 217 ± 7 | 446 ± 43 | 865 ± 87 | NA | NA |
| G2 | 217 ± 9 | 266 ± 27 | 640 ± 61 | 34.7 | 0.067 |
| G3 | 217 ± 7 | 154 ± 14 | 282 ± 27 | 90.0 | 2.15E−04 |
| G4 | 217 ± 8 | 175 ± 20 | 357 ± 40 | 78.4 | 0.001 |
| G5 | 217 ± 9 | 253 ± 21 | 565 ± 129 | 46.3 | 0.091 |
| G6 | 217 ± 15 | 268 ± 32 | 543 ± 42 | 49.7 | 0.010 |
| G7 | 217 ± 7 | 419 ± 78 | 1008 ± 161 | −22.1 | 0.458 |

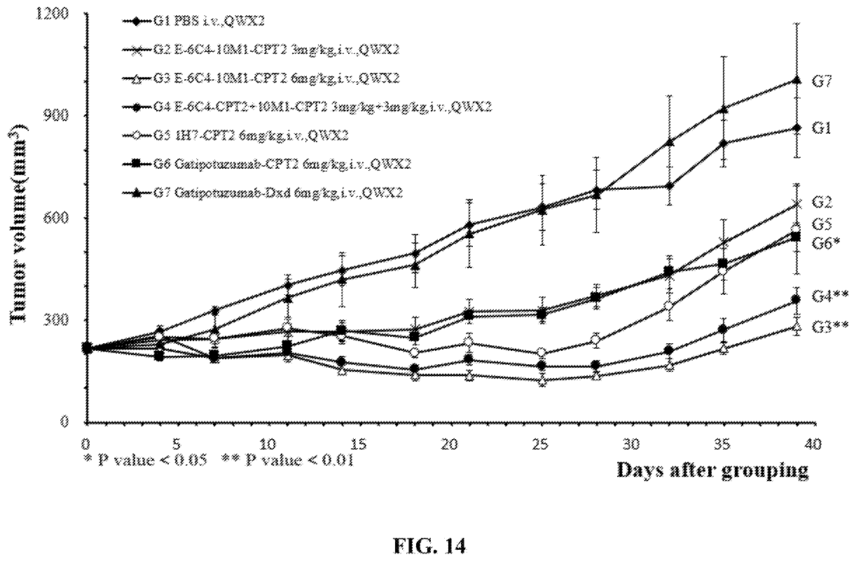

The tumor volume of mice in different groups are shown in FIG. 14. The results showed that E-6C4-10M1-CPT2 exhibited tumor inhibitory effects in a dose-dependent manner, and had better tumor inhibitory effects than the combination of the parental monoclonal ADCs E-6C4-CPT2 and 10M1-CPT2. In addition, E-6C4-OM1-CPT2 also exhibited better tumor inhibitory effect than the positive controls 1H7-CPT2, Gatipotuzumab-CPT2, and Gatipotuzumab-Dxd.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An anti-EGFR/MUC1 antibody or antigen-binding fragment thereof, comprising: a first antigen-binding domain that specifically binds to EGFR; and a second antigen-binding domain that specifically binds to MUC1.

2. The anti-EGFR/MUC1 antibody or antigen-binding fragment thereof of claim 1, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

3. The anti-EGFR/MUC1 antibody or antigen-binding fragment thereof of claim 2, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively; and
(10) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively.

4. The anti-EGFR/MUC1 antibody or antigen-binding fragment thereof of claim 2 or 3, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively; and
(4) the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

5. The anti-EGFR/MUC1 antibody or antigen-binding fragment thereof of any one of claims 2-4, wherein
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 40-42, respectively, and the selected VL2 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(18) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(19) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively; or
(20) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

6. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

7. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

8. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

9. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

10. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 52, and the second light chain variable region comprises a sequence that is at least 80%, 85%, 90%, 95%, 99%, or 100% identical to SEQ ID NO: 46.

11. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

12. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 48, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

13. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 49, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

14. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 50, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

15. The anti-EGFR/MUC1 antibody or antigen-binding fragment thereof of any one of claims 2-5, wherein the first heavy chain variable region comprises a sequence that is at least 80%, 85%, 90%, 95%, 99%, or 100% identical to SEQ ID NO: 51, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 53, and the second light chain variable region comprises a sequence that is at least 80%, 85%, 90%, 95%, 99%, or 100% identical to SEQ ID NO: 46.

16. The anti-EGFR/MUC1 antibody or antigen-binding fragment thereof of any one of claims 2-15, wherein the VH1 comprises an amino acid sequence that is at least 90% identical to a selected VH sequence, and the VL 1 comprises an amino acid sequence that is at least 90% identical to a selected VL sequence, wherein the selected VH sequence and the selected VL sequence are one of the following:
(1) the selected VH sequence is SEQ ID NO: 47, and the selected VL sequence is SEQ ID NO: 46;
(2) the selected VH sequence is SEQ ID NO: 48, and the selected VL sequence is SEQ ID NO: 46;
(3) the selected VH sequence is SEQ ID NO: 49, and the selected VL sequence is SEQ ID NO: 46;
(4) the selected VH sequence is SEQ ID NO: 50, and the selected VL sequence is SEQ ID NO: 46; and
(5) the selected VH sequence is SEQ ID NO: 51, and the selected VL sequence is SEQ ID NO: 46.

17. The anti-EGFR/MUC1 antibody or antigen-binding fragment thereof of any one of claims 2-16, wherein the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 47, and the selected VL sequence is SEQ ID NO: 46;
(2) the selected VH sequence is SEQ ID NO: 48, and the selected VL sequence is SEQ ID NO: 46;
(3) the selected VH sequence is SEQ ID NO: 49, and the selected VL sequence is SEQ ID NO: 46;
(4) the selected VH sequence is SEQ ID NO: 50, and the selected VL sequence is SEQ ID NO: 46; and
(5) the selected VH sequence is SEQ ID NO: 51, and the selected VL sequence is SEQ ID NO: 46.

18. The anti-EGFR/MUC1 antibody or antigen-binding fragment thereof of any one of claims 2-17, wherein the VH2 comprises an amino acid sequence that is at least 90% identical to a selected VH sequence, and the VL2 comprises an amino acid sequence that is at least 90% identical to a selected VL sequence, wherein the selected VH sequence and the selected VL sequence are one of the following:
(1) the selected VH sequence is SEQ ID NO: 52, and the selected VL sequence is SEQ ID NO: 46; and
(2) the selected VH sequence is SEQ ID NO: 53, and the selected VL sequence is SEQ ID NO: 46.

19. The anti-EGFR/MUC1 antibody or antigen-binding fragment thereof of any one of claims 2-18, wherein the VH2 comprises VH2 CDR1, VH2 CDR2, and VH2 CDR3 that are identical to VH CDR1, VH CDR2, and VH CDR3 of a selected VH sequence; and the VL2 comprising VL2 CDR 1, VL2 CDR2, and VL2 CDR3 that are identical to VL CDR 1, VL CDR2, and VL CDR3 of a selected VL sequence, wherein the selected VH sequence and the selected VL sequence are one of the following:
(1) the selected VH sequence is SEQ ID NO: 52, and the selected VL sequence is SEQ ID NO: 46; and
(2) the selected VH sequence is SEQ ID NO: 53, and the selected VL sequence is SEQ ID NO: 46.

20. The anti-EGFR/MUC1 antibody or antigen-binding fragment thereof of any one of claims 2-19, wherein the VH1 comprises the sequence of SEQ ID NO: 47 and the VL1 comprises the sequence of SEQ ID NO: 46.

21. The anti-EGFR/MUC1 antibody or antigen-binding fragment thereof of any one of claims 2-19, wherein the VH1 comprises the sequence of SEQ ID NO: 48 and the VL1 comprises the sequence of SEQ ID NO: 46.

22. The anti-EGFR/MUC1 antibody or antigen-binding fragment thereof of any one of claims 2-19, wherein the VH1 comprises the sequence of SEQ ID NO: 49 and the VL1 comprises the sequence of SEQ ID NO: 46.

23. The anti-EGFR/MUC1 antibody or antigen-binding fragment thereof of any one of claims 2-19, wherein the VH1 comprises the sequence of SEQ ID NO: 50 and the VL1 comprises the sequence of SEQ ID NO: 46.

24. The anti-EGFR/MUC1 antibody or antigen-binding fragment thereof of any one of claims 2-19, wherein the VH1 comprises the sequence of SEQ ID NO: 51 and the VL1 comprises the sequence of SEQ ID NO: 46.

25. The anti-EGFR/MUC1 antibody or antigen-binding fragment thereof of any one of claims 2-24, wherein the VH2 comprises the sequence of SEQ ID NO: 52 and the VL2 comprises the sequence of SEQ ID NO: 46.

26. The anti-EGFR/MUC1 antibody or antigen-binding fragment thereof of any one of claims 2-24, wherein the VH2 comprises the sequence of SEQ ID NO: 53 and the VL2 comprises the sequence of SEQ ID NO: 46.

27. The anti-EGFR/MUC1 antibody or antigen-binding fragment thereof of any one of claims 1-26, wherein the first antigen-binding domain specifically binds to human or monkey EGFR; and/or the second antigen-binding domain specifically binds to human or monkey MUC1.

28. The anti-EGFR/MUC1 antibody or antigen-binding fragment thereof of any one of claims 1-27, wherein the first antigen-binding domain is human or humanized; and/or the second antigen-binding domain is human or humanized.

29. The anti-EGFR/MUC1 antibody or antigen-binding fragment thereof of any one of claims 1-28, wherein the antibody is a bispecific antibody.

30. The anti-EGFR/MUC1 antibody or antigen-binding fragment thereof of any one of claims 1-29, wherein the first antigen-binding domain is a single-chain variable fragment (scFv); and/or the second antigen-binding domain is a scFv.

31. The anti-EGFR/MUC1 antibody or antigen-binding fragment thereof of any one of claims 2-30, wherein the first light chain variable region and the second light chain variable region are identical.

32. An anti-EGFR/MUC1 antibody or antigen-binding fragment thereof that cross-competes with the anti-EGFR/MUC1 antibody or antigen-binding fragment thereof of any one of claims 1-31.

33. A nucleic acid comprising a polynucleotide encoding the anti-EGFR/MUC1 antibody or antigen-binding fragment thereof of any one of claims 1-32.

34. A vector comprising the nucleic acid of claim 33.

35. A cell comprising the vector of claim 34.

36. The cell of claim 35, wherein the cell is a CHO cell.

37. A cell comprising the nucleic acid of claim 33.

38. A method of producing an anti-EGFR/MUC1 antibody or an antigen-binding fragment thereof, the method comprising
(a) culturing the cell of any one of claims 35-37 under conditions sufficient for the cell to produce the anti-EGFR/MUC1 antibody or the antigen-binding fragment thereof; and
(b) collecting the anti-EGFR/MUC1 antibody or the antigen-binding fragment thereof produced by the cell.

39. An anti-EGFR/MUC1 antibody-drug conjugate (ADC) comprising a therapeutic agent covalently bound to the anti-EGFR/MUC1 antibody or antigen-binding fragment thereof of any one of claims 1-32.

40. The anti-EGFR/MUC1 antibody drug conjugate of claim 39, wherein the therapeutic agent is a cytotoxic or cytostatic agent.

41. The anti-EGFR/MUC1 antibody drug conjugate of claim 39 or 40, wherein the therapeutic agent is MMAE or MMAF.

42. The antibody-drug conjugate of claim 39, wherein the therapeutic agent is selected from

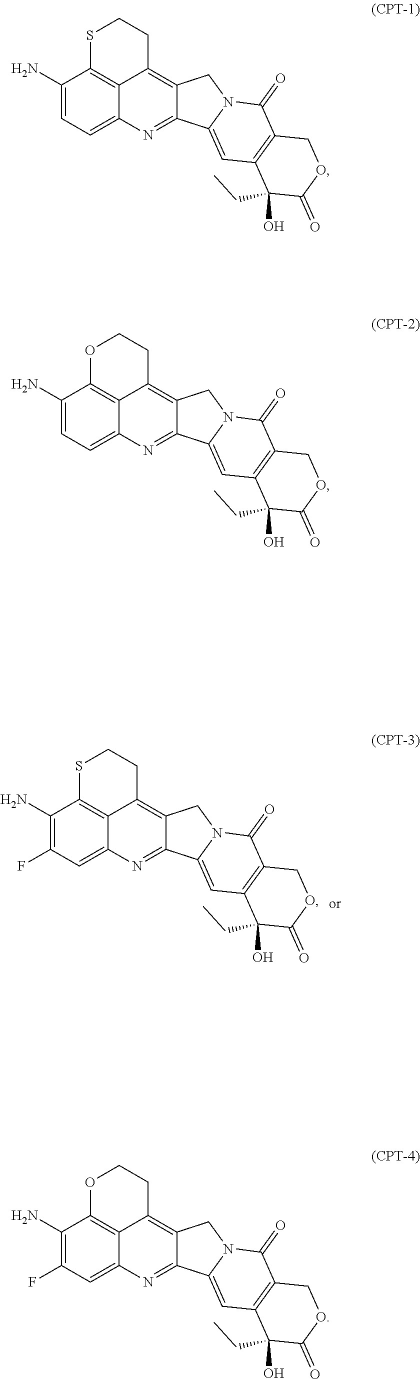 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

43. The antibody-drug conjugate of claim 39 or 42, wherein the therapeutic agent is linked to the antibody or antigen-binding fragment thereof via a linker.

44. The antibody-drug conjugate of claim 43, wherein the linker has a structure of:

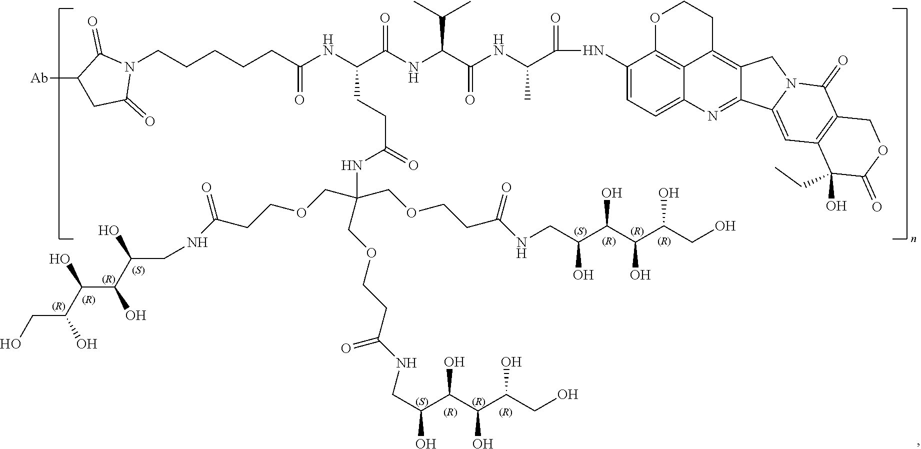

45. The antibody-drug conjugate of any one of claims 39 and 42-44, wherein the antibody-drug conjugate has a structure of: 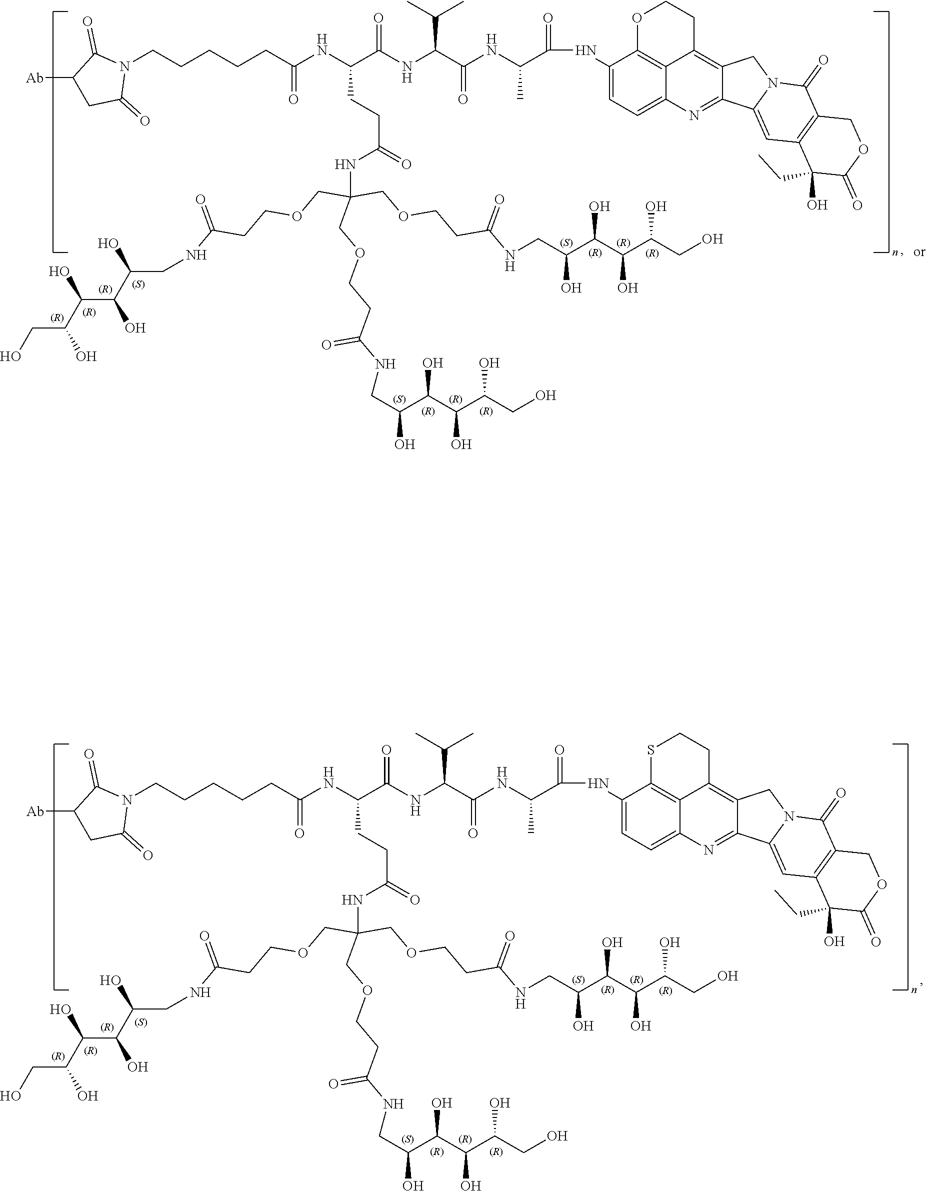
wherein n=1-8; wherein “Ab” represents the antibody or antigen-binding fragment thereof.

46. A method of treating a subject having cancer, the method comprising administering a therapeutically effective amount of a composition comprising the anti-EGFR/MUC1 antibody or antigen-binding fragment thereof of any one of claims 1-32, or the anti-EGFR/MUC1 antibody-drug conjugate of any one of claims 39-45, to the subject.

47. The method of claim 46, wherein the subject has a cancer expressing EGFR and/or MUC1 (e.g., both EGFR and MUC1).

48. The method of claim 46 or claim 47, wherein the cancer is solid tumor, lung cancer, head and neck cancer, colon cancer, pancreatic cancer, breast cancer, ovarian cancer, bladder cancer, kidney cancer, gliomas, oesophageal cancer, gastric cancer, stomach cancer, prostate cancer, cervical cancer, multiple myeloma, or Non-Hodgkin Lymphoma.

49. The method of any one of claims 46-48, wherein the subject is a human.

50. The method of any one of claims 46-49, wherein the method further comprises administering an anti-PD1 antibody to the subject.

51. The method of any one of claims 46-50, wherein the method further comprises administering a chemotherapy to the subject.

52. A method of decreasing the rate of tumor growth, the method comprising contacting a tumor cell with an effective amount of a composition comprising the anti-EGFR/MUC1 antibody or antigen-binding fragment thereof of any one of claims 1-32, or the anti-EGFR/MUC1 antibody-drug conjugate of any one of claims 39-45.

53. A method of killing a tumor cell, the method comprising contacting a tumor cell with an effective amount of a composition comprising the anti-EGFR/MUC1 antibody or antigen-binding fragment thereof of any one of claims 1-32, or the anti-EGFR/MUC1 antibody-drug conjugate of any one of claims 39-45.

54. A pharmaceutical composition comprising a pharmaceutically acceptable carrier and
(a) the anti-EGFR/MUC 1 antibody or antigen-binding fragment thereof of any one of claims 1-32, and/or
(b) the anti-EGFR/MUC1 antibody-drug conjugate of any one of claims 39-45.

55. The anti-EGFR/MUC1 ADC of any one of claims 39-45, wherein the drug-to-antibody ratio (DAR) is about 4 or 8.


SEQUENCE LISTING (67 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 7 aa, synthetic construct
SGDYYWS

SEQ ID NO: 5 - protein, 16 aa, synthetic construct
YIYYSGSTYYNPSLKS

SEQ ID NO: 6 - protein, 10 aa, synthetic construct
ERVYSSSLDY

SEQ ID NO: 7 - protein, 7 aa, synthetic construct
SGDYYWN

SEQ ID NO: 8 - protein, 16 aa, synthetic construct
YIYYSGSTYYNPSLKS

SEQ ID NO: 9 - protein, 10 aa, synthetic construct
EGVYTIMVDY

SEQ ID NO: 10 - protein, 7 aa, synthetic construct
SGDYYWS

SEQ ID NO: 11 - protein, 16 aa, synthetic construct
YIYYSGSTYYNPSLKS

SEQ ID NO: 12 - protein, 10 aa, synthetic construct
DLVYYYALDV

SEQ ID NO: 13 - protein, 7 aa, synthetic construct
SGDYYWS

SEQ ID NO: 14 - protein, 16 aa, synthetic construct
HIYYSGITYYNPSLKS

SEQ ID NO: 15 - protein, 9 aa, synthetic construct
AGVYHDMDV

SEQ ID NO: 16 - protein, 7 aa, synthetic construct
SGDYYWS

SEQ ID NO: 17 - protein, 16 aa, synthetic construct
YIYYSGSTYYNPSLKS

SEQ ID NO: 18 - protein, 10 aa, synthetic construct
VGVYSTSRDY

SEQ ID NO: 19 - protein, 5 aa, synthetic construct
SYGMH

SEQ ID NO: 20 - protein, 17 aa, synthetic construct
VISYDGSNKYYADSVKG

SEQ ID NO: 21 - protein, 18 aa, synthetic construct
DRRGFYESGNYYNVPFDY

SEQ ID NO: 22 - protein, 5 aa, synthetic construct
SYAMH

SEQ ID NO: 23 - protein, 17 aa, synthetic construct
VISDDGINKYYADSVKG

SEQ ID NO: 24 - protein, 18 aa, synthetic construct
ALPTIFGVVMYFYYGLDV

SEQ ID NO: 25 - protein, 9 aa, synthetic construct
GGSINSGDY

SEQ ID NO: 26 - protein, 5 aa, synthetic construct
YYSGS

SEQ ID NO: 27 - protein, 10 aa, synthetic construct
ERVYSSSLDY

SEQ ID NO: 28 - protein, 9 aa, synthetic construct
GGSISSGDY

SEQ ID NO: 29 - protein, 5 aa, synthetic construct
YYSGS

SEQ ID NO: 30 - protein, 10 aa, synthetic construct
EGVYTIMVDY

SEQ ID NO: 31 - protein, 9 aa, synthetic construct
GGSISSGDY

SEQ ID NO: 32 - protein, 5 aa, synthetic construct
YYSGS

SEQ ID NO: 33 - protein, 10 aa, synthetic construct
DLVYYYALDV

SEQ ID NO: 34 - protein, 9 aa, synthetic construct
GGSINSGDY

SEQ ID NO: 35 - protein, 5 aa, synthetic construct
YYSGI

SEQ ID NO: 36 - protein, 9 aa, synthetic construct
AGVYHDMDV

SEQ ID NO: 37 - protein, 9 aa, synthetic construct
GDSISSGDY

SEQ ID NO: 38 - protein, 5 aa, synthetic construct
YYSGS

SEQ ID NO: 39 - protein, 10 aa, synthetic construct
VGVYSTSRDY

SEQ ID NO: 40 - protein, 7 aa, synthetic construct
GFTFSSY

SEQ ID NO: 41 - protein, 6 aa, synthetic construct
SYDGSN

SEQ ID NO: 42 - protein, 18 aa, synthetic construct
DRRGFYESGNYYNVPFDY

SEQ ID NO: 43 - protein, 7 aa, synthetic construct
GFTFSSY

SEQ ID NO: 44 - protein, 6 aa, synthetic construct
SDDGIN

SEQ ID NO: 45 - protein, 18 aa, synthetic construct
ALPTIFGVVMYFYYGLDV

SEQ ID NO: 46 - protein, 107 aa, synthetic construct [sequence omitted]

SEQ ID NO: 47 - protein, 120 aa, synthetic construct
QVQLQQWGPGLVKPSQTLSLTCTVSGGSINSGDYYWSWIRQPPGKGLESIGYIYYSGSTYYNPSLKSRVTISADTSKNQFSLKLTSVTAADTAVYYCARERVYSSSLDYWGQGTLVTVSS

SEQ ID NO: 48 - protein, 120 aa, synthetic construct
EVQLQQSGPGLVKPSQTLSLTCTVSGGSISSGDYYWNWIRQPPGKGLEWIGYIYYSGSTYYNPSLKSRVTISVDTSKNQFSLKLSSVTAADTAVYYCAREGVYTIMVDYWGQGTLVTVSS

SEQ ID NO: 49 - protein, 120 aa, synthetic construct
EVQLQESGPGLVKPSQTLSLTCTVSGGSISSGDYYWSWIRQPPGKGLEWIGYIYYSGSTYYNPSLKSRVTISVDTSKNQFSLRLSSVTAADTAVYYCARDLVYYYALDVWGQGTTVTVSS

SEQ ID NO: 50 - protein, 119 aa, synthetic construct
QVQLQESGPGLVKPSQTLSLTCTVSGGSINSGDYYWSWIRQPPGKGLEWIGHIYYSGITYYNPSLKSRVTISADTSKNQFSLKLTSVTVADTAVYYCDRAGVYHDMDVWGQGTTVTVSS

SEQ ID NO: 51 - protein, 120 aa, synthetic construct
EVQLVESGPGLVKPSQTLSLTCTVSGDSISSGDYYWSWIRQPPGKGLEWIGYIYYSGSTYYNPSLKSRVIISLDTSKNQFSLKLISVTAADTAVYYCARVGVYSTSRDYWGQGTLVTVSS

SEQ ID NO: 52 - protein, 127 aa, synthetic construct
QVTLKESGGGVVQPGRSLRLSCAASGFTFSSYGMHWVRQAPGKGLEWVAVISYDGSNKYYADSVKGRFTISRDNSKNTLYLQMNSLRAEDTAVYYCANDRRGFYESGNYYNVPFDYWGQGTLVTVSS

SEQ ID NO: 53 - protein, 127 aa, synthetic construct
QVQLVQSGGGVVQPGRSLRLSCAASGFTFSSYAMHWVRQAPGKGLEWVAVISDDGINKYYADSVKGRFTISRDNSKNTLYLQMNSLRAEDSAVYYCARALPTIFGVVMYFYYGLDVWGQGTTATVSS

SEQ ID NO: 54 - protein, 107 aa, synthetic construct [sequence omitted]

SEQ ID NO: 55 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 621 aa, Homo sapiens [sequence omitted]

SEQ ID NO: 58 - protein, 621 aa, Macaca fascicularis
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPS

SEQ ID NO: 59 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 60 - protein, 200 aa, Macaca fascicularis
ASDSASGSASTLVHSTTSARATTTPASKSTPFSIPSHHSDTPTTLASHSTKTDASSTHHSTVPPFTSSNHSTSPQLSLGVSFFFLSFHISNLQFNSSLEDPSTNYYQQLQRDISELFLQIYKQGDFLGLSNIMFRPGSVVVQSTLVFREGTTNVHDVETQFNQRKTEAASRYNLTISDISVRDVPFPFSAQTGAGVPGWG

SEQ ID NO: 61 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 116 aa, synthetic construct
EVQLQQSGAELAKPGASVKMSCKVSGYTFTSYWMHWVKQRPGQGLEWIGYINPGTGYIEYNQKFKDKATLTADKSSSTAYMQLSSLTSEDSAVYYCASSTAPFDYWGQGTTLTVSS

SEQ ID NO: 64 - protein, 107 aa, synthetic construct
DIVITQSPSSMYASLGERVTITCKASQDIKSYLSWYQQKPWKSPKTLIYYATRLADGVPSRFSGSGSGQDYSLTISSLESDDTATYYCLQYDESPYTFGGGTKLEIK

SEQ ID NO: 65 - protein, 117 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 66 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 67 - protein, 364 aa, Homo sapiens
PKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG